(19) United States
(12) Patent Application Publication
(10) Pub. No.: US 2026/0220336 A1
(43) Pub. Date: Jul. 30, 2026

(54) SYSTEMS AND METHODS FOR PREDICTION OF ANTIBIOTIC RESISTANCE FROM BACTERIAL GENOMES

(71) Applicant: THE BOARD OF REGENTS OF THE UNIVERSITY OF TEXAS SYSTEM, Austin, TX (US)

(72) Inventors: Xiaowei Zhan, Dallas, TX (US); David E. Greenberg, Dallas, TX (US)

(21) Appl. No.: 19/124,177

(22) PCT Filed: Oct. 25, 2023
(86) PCT No.: PCT/US2023/077718

§ 371 Date: Apr. 24, 2025

Related U.S. Application Data
(60) Provisional application No. 63/381,086, filed on Oct. 26, 2022.

(51) Int. Cl. G06F 30/27

(52) U.S. Cl. CPC G06F 30/27

(57) ABSTRACT

Systems, methods, and devices disclosed herein provide antibiotic resistance predictions using a pan-antibiotic resistance prediction (PARP) model. The PARP model includes a machine learning system trained with a training data set of genetic information associated with a plurality of bacterial species, and/or antibiotic feature information associated with a plurality of antibiotics. The PARP model is deployed to a cloud-based service for scalability which provides access to the PARP model for clinic devices, hospital devices, and/or laboratory devices. For instance, a web-based portal of the cloud-based service receives a genomic sequence associated with a particular bacterial isolate, uploaded via a remote device. The PARP model outputs a predictive indication of an antibiotic resistance, for the particular bacterial isolate. The predictive indication can include a bar graph (e.g., presented at a graphical user interface) showing, for the particular bacterial isolate, susceptibility/resistance predictions for a plurality of antibiotics.

### CROSS-REFERENCE TO RELATED APPLICATION

This application claims priority to U.S. Provisional Patent Application Ser. No. 63/381,086 filed on Oct. 26, 2022 and titled “SYSTEMS AND METHODS FOR PREDICTION OF ANTIBIOTIC RESISTANCE FROM BACTERIAL GENOMES,” the entirety of which is incorporated herein by reference.

### ACKNOWLEDGEMENT OF GOVERNMENT SUPPORT

This invention was made with government support under grant number A1169298 awarded by the National Institutes of Health, and grant numbers W81XWH-20-1-0149, PR192594 awarded by the United States Department of Defense. The government has certain rights in this invention.

### BACKGROUND

### 1. Technical Field

Aspects of the presently disclosed technology generally relate to systems and methods for predicting antibiotic resistance, and more specifically, for predicting antibiotic resistance based on genomic information.

### 2. Discussion of Related Art

Antibiotic Resistance (AR) is a public health threat. Each year in the United States, at least 2.8 million people are infected with antibiotic-resistant bacteria or fungi, and more than 35,000 people die as a result. The acquisition of resistance by pathogens leads to challenges in providing effective therapy, resulting in prolonged hospital stays, expensive alternative therapies, and increased mortality rates. Financially, the total economic burden for AR infection can be up to $20 billion in health care and $35 billion in loss of productivity annually. Conventional diagnostic methods rely on culture followed by antibiotic susceptibility testing (AST), which can take days to weeks to complete.

### SUMMARY

Systems, methods, and devices disclosed herein can address the aforementioned issues. For instance, a method for antibiotic resistance prediction can include receiving, at a machine learning system, genetic information associated with a bacteria, a species of the bacteria being one of a plurality of bacterial species; receiving, at the machine learning system, an indication of an antibiotic of a plurality of antibiotics, wherein the machine learning system is trained using genetic information associated with the plurality of bacterial species and for the plurality of antibiotics; and/or outputting, at the machine learning system, an indication of an antibiotic resistance or susceptibility associated with the bacteria and the antibiotic received at the machine learning system.

In some examples, the machine learning system can be trained using protein sequences associated with the plurality of bacterial species. The machine learning system can include a feature-wise linear modulation (FiLM) machine learning system. Additionally, the machine learning system can jointly model antibiotics and bacterial variants. Furthermore, the machine learning system can include a rectified linear activation function (ReLU) layer, a batch normalization layer, and/or a dropout layer.

In some examples, a method for antibiotic resistance prediction includes training a pan-antibiotic resistance prediction (PARP) model by providing a machine learning system with one or more training data sets including genetic information associated with a plurality of bacterial species, and/or antibiotic feature information associated with a plurality of antibiotics. The method can also include receiving, at the machine learning system, a genomic sequence associated with a particular bacterial isolate; and/or outputting, at the machine learning system, a predictive indication of an antibiotic resistance, associated with one or more antibiotics, for the particular bacterial isolate.

In some examples, the method further includes performing a data preparation procedure on the one or more training data sets by one-hot encoding the antibiotic feature information. Additionally, the one or more training data sets can include at least one of an isolates-variants matrix, an antibiotics indicator matrix, or an isolates resistance symptom vector. The method can also include performing a nested cross-validation procedure on the PARP model using a validation data set including a plurality of bacteria-antibiotic combinations. Furthermore, the method can include determining, with the PARP model, one or more classes associated with the plurality of antibiotics, or the plurality of bacterial species, using weights of one or more dense layers of a Feature wise Linear Modulator (FiLM) generator to form clusters, wherein the machine learning system uses the one or more classes to output the predictive indication of the antibiotic resistance. Also, the PARP model can be deployed onto a container orchestration service such that the PARP model provides a cloud-based antibiotics resistance prediction service.

In some examples, receiving the genomic sequence can include receiving an upload, from a remote device, at the cloud-based antibiotics resistance prediction service. The genomic sequence can correspond to a bacterial species absent from the one or more training data sets. Furthermore, the predictive indication of the antibiotic resistance can include a bar graph for presentation at a graphical user interface (GUI) of a computing device that provided the genomic sequence. Moreover, an x-axis of the bar graph can represent different antibiotics and a y-axis of the bar graph can represent a prediction value of resistance or susceptibility to the different antibiotics. The PARP model can generate shared and unique variant data indicating one or more variants shared between different bacteria species and one or more variants unique to the different bacteria species. Training the PARP model can include generating paired-antibiotic susceptibility data based on tests of isolates on antibiotic pairs indicating shared pathways of the antibiotic pairs. The method can also include performing a prediction accuracy assessment for the predictive indication, the prediction accuracy assessment outputs one or more prediction accuracy values corresponding to the one or more antibiotics. Furthermore, the method can include tuning a plurality of hyperparameters of the PARP model, the plurality of hyperparameters includes a number of dense blocks, a number of layers or feature stacking blocks, and/or a geometric size of a dense layer.

In some examples, a system for antibiotic resistance prediction includes a pan-antibiotic resistance prediction (PARP) model deployed to a cloud-based service, the PARP model having a machine learning system trained with one or more training data sets including: genetic information associated with a plurality of bacterial species, and/or antibiotic feature information associated with a plurality of antibiotics. The system can also include a web-based portal for receiving a genomic sequence associated with a particular bacterial isolate and providing the genomic sequence to the PARP model; and/or a predictive indication of an antibiotic resistance, for the particular bacterial isolate, outputted by the PARP model and configured for presentation at a graphical user interface (GUI) of a computing device.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 illustrates an example computing device, in accordance with certain aspects of the presently disclosed technology.

FIG. 2 illustrates an example antibiotic resistance prediction system implemented using a machine learning model, in accordance with certain aspects of the present disclosure.

FIG. 3 is a block diagram illustrating an example machine learning system, in accordance with certain aspects of the present disclosure.

FIG. 4 is a flow diagram illustrating example operations for antibiotic resistance prediction, in accordance with certain aspects of the presently disclosed technology.

FIG. 5A illustrates an example antibiotic resistance prediction system workflow, in accordance with certain aspects of the presently disclosed technology.

FIG. 5B illustrates an example antibiotic resistance heat map of an antibiotic resistance prediction system, in accordance with certain aspects of the presently disclosed technology.

FIG. 5C illustrates an example variants graph of an antibiotic resistance prediction system, in accordance with certain aspects of the presently disclosed technology.

FIGS. 6A and 6B illustrate an example prediction accuracy evaluation of an antibiotic resistance prediction system, in accordance with certain aspects of the presently disclosed technology.

FIG. 7A illustrates an example classification graph of an antibiotic resistance prediction system, in accordance with certain aspects of the presently disclosed technology.

FIG. 7B illustrates an example agglomerative clustering dendrogram of an antibiotic resistance prediction system, in accordance with certain aspects of the presently disclosed technology.

FIG. 7C illustrates an example classification graph of an antibiotic resistance prediction system, in accordance with certain aspects of the presently disclosed technology.

FIG. 7D illustrates an example agglomerative clustering dendrogram of an antibiotic resistance prediction system, in accordance with certain aspects of the presently disclosed technology.

FIG. 8A illustrates an example cloud-based deployment of an antibiotic resistance prediction system, in accordance with certain aspects of the presently disclosed technology.

FIG. 8B illustrates an example bar graph of prediction output results of an antibiotic resistance prediction system, in accordance with certain aspects of the presently disclosed technology.

FIGS. 9A and 9B illustrate example unique/shared variant bar graphs of an antibiotic resistance prediction system, in accordance with certain aspects of the presently disclosed technology.

FIGS. 10A-10D illustrate a plurality of example unique/shared variant bar graphs of an antibiotic resistance prediction system, in accordance with certain aspects of the presently disclosed technology.

FIG. 11 illustrates an antibiotic assessment engine of an antibiotic resistance prediction system, in accordance with certain aspects of the presently disclosed technology.

FIGS. 12A and 12B illustrate an example nested cross-validation procedure of an antibiotic resistance prediction system, in accordance with certain aspects of the presently disclosed technology.

FIG. 13 illustrates an example prediction output comparison of an antibiotic resistance prediction system, in accordance with certain aspects of the presently disclosed technology.

FIG. 14 illustrates an example prediction accuracy assessment of an antibiotic resistance prediction system, in accordance with certain aspects of the presently disclosed technology.

FIG. 15 illustrates an example prediction accuracy assessment of an antibiotic resistance prediction system, in accordance with certain aspects of the presently disclosed technology.

FIG. 16 illustrates an example antibiotic resistance prediction system having a Feature-wise Linear Modulation (FiLM) generator, in accordance with certain aspects of the presently disclosed technology.

It will be apparent to one skilled in the art after review of the entirety disclosed that the steps illustrated in the figures listed above may be performed in other than the recited order, and that one or more steps illustrated in these figures may be optional.

### DETAILED DESCRIPTION

Certain aspects of the presently disclosed technology are directed to methods and systems for predicting antibiotic resistance based on genomic information. The antibiotic resistance prediction system described herein may include a machine learning system for in-silico antibiotic resistance determination. The development of the machine learning system can be based on curation of bacterial isolates (e.g., over 3000 bacterial isolates) and different antibiotics (e.g., 29 antibiotics). The machine learning system can provide a high prediction performance by using an advanced deep learning algorithm. The antibiotic resistance prediction system provides scalability and affordability as a cloud-native solution. Moreover, the antibiotic resistance prediction system can be a pathogen agnostic predictive algorithm to predict antibiotic resistance for any genome-sequenced pathogen, even those not included in the training data set.

Additional benefits and advantages of the disclosed technology will become apparent from the detailed description below.

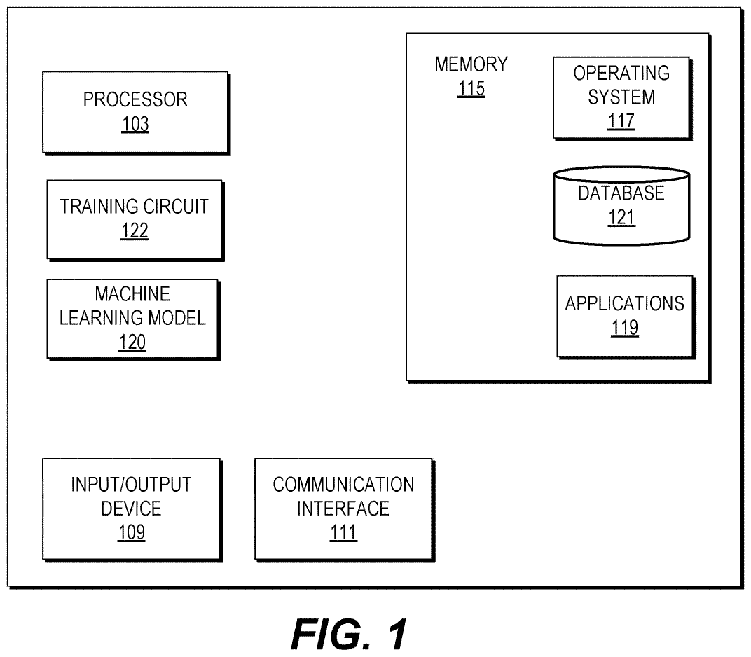

FIG. 1 illustrates an example computing device 100, in accordance with certain aspects of the presently disclosed technology. The computing device 100 can include a processor 103 for controlling overall operation of the computing device 100 and its associated components, including input/output device 109, communication interface 111, and/or memory 115. A data bus can interconnect processor(s) 103, memory 115, I/O device 109, and/or communication interface 111.

Input/output (I/O) device 109 can include a microphone, keypad, touch screen, and/or stylus through which a user of the computing device 100 can provide input and can also include one or more of a speaker for providing audio output and a video display device for providing textual, audiovisual, and/or graphical output. Software can be stored within memory 115 to provide instructions to processor 103 allowing computing device 100 to perform various actions. For example, memory 115 can store software used by the computing device 100, such as an operating system 117, application programs 119, and/or an associated internal database 121. The various hardware memory units in memory 115 can include volatile and nonvolatile, removable, and non-removable media implemented in any method or technology for storage of information such as computer-readable instructions, data structures, program modules or other data. Memory 115 can include one or more physical persistent memory devices and/or one or more non-persistent memory devices. Memory 115 can include, but is not limited to, random access memory (RAM), read only memory (ROM), electronically erasable programmable read only memory (EEPROM), flash memory or other memory technology, CD-ROM, digital versatile disks (DVD) or other optical disk storage, magnetic cassettes, magnetic tape, magnetic disk storage or other magnetic storage devices, or any other medium that can be used to store the desired information and that can be accessed by processor 103.

Communication interface 111 can include one or more transceivers, digital signal processors, and/or additional circuitry and software for communicating via any network, wired or wireless, using any protocol as described herein. Processor 103 can include a single central processing unit (CPU), which can be a single-core or multi-core processor (e.g., dual-core, quad-core, etc.), or can include multiple CPUs. Processor(s) 103 and associated components can allow the computing device 100 to execute a series of computer-readable instructions to perform some or all of the processes described herein. Although not shown in FIG. 1, various elements within memory 115 or other components in computing device 100, can include one or more caches, for example, CPU caches used by the processor 103, page caches used by the operating system 117, disk caches of a hard drive, and/or database caches used to cache content from database 121. For implementations including a CPU cache, the CPU cache can be used by one or more processors 103 to reduce memory latency and access time. A processor 103 can retrieve data from or write data to the CPU cache rather than reading/writing to memory 115, which can improve the speed of these operations. In some examples, a database cache can be created in which certain data from a database 121 is cached in a separate smaller database in a memory separate from the database, such as in RAM or on a separate computing device. For instance, in a multi-tiered application, a database cache on an application server can reduce data retrieval and data manipulation time by not needing to communicate over a network with a back-end database server. These types of caches and others can be included in various implementations and can provide potential advantages in certain implementations of software deployment systems, such as faster response times and less dependence on network conditions when transmitting and receiving data.

In certain aspects of the present disclosure, the computing device 100 may include a machine learning model 120. The machine learning model 120 may be implemented as part of the processor 103, in some implementations. The machine learning model 120 may be trained using a training circuit 122. The machine learning model 120 may be trained to predict antibiotic resistance based on genomic information across various bacterial species, thus forming the pan-antibiotic resistance prediction (PARP) model 504, discussed in greater detail below. In some aspects, the computing device 100 may be implemented on a network (e.g., on a server), to implement antibiotic resistance prediction on the cloud.

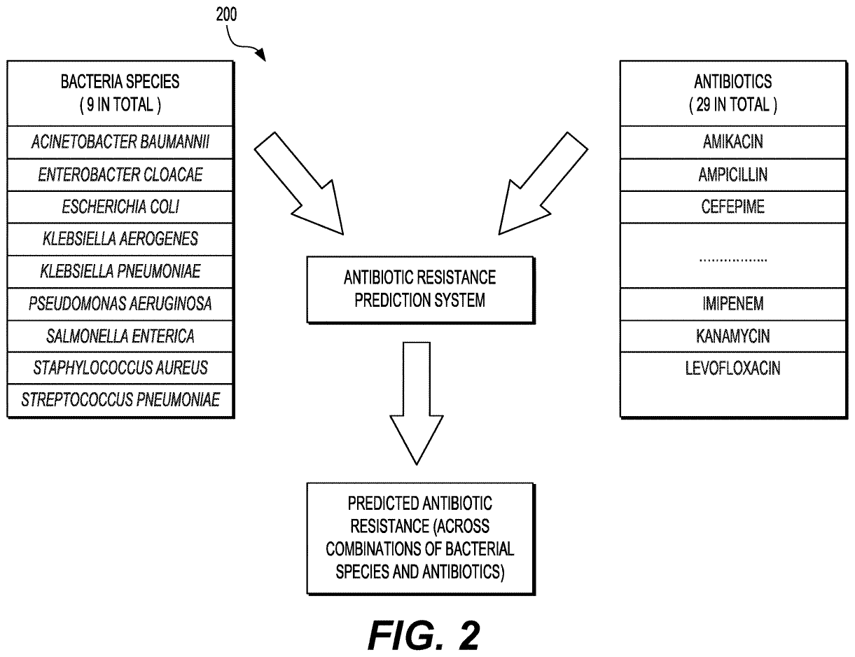

FIG. 2 illustrates an example antibiotic resistance prediction system 200 implemented using a machine learning model 120, in accordance with certain aspects of the present disclosure. The antibiotic resistance prediction system 200 may be implemented on the cloud, providing an interface for users predict antibiotic resistance by interacting with the antibiotic resistance prediction system 200.

Genomic information for any bacteria (e.g., across various bacterial species) may be provided to the antibiotic resistance prediction system 200. In some aspects, a particular antibiotic may be provided to the antibiotic resistance prediction system 200. Using a trained machine learning model (machine learning system), the antibiotic resistance prediction system 200 may predict a level of resistance by the bacteria to the antibiotic provided to the antibiotic resistance prediction system 200. In some aspects, the input to the machine learning model may be a consensus protein sequence of a translated DNA for the bacteria and the characterization/definition of protein variants.

To train the machine learning model, entire bacterial genomes of various bacterial species may be provided for training. In some cases, specific antibiotic resistance genes may be used to train the model. The trained machine learning model may thus provide a resistance prediction based on an input of any genome of any pathogen (e.g., the model is not limited to a particular bacterial species or pathogen and can provide a resistance prediction for any bacteria of any bacterial species input to the model). The machine learning model predicts resistance for multiple antibiotics and may identify new potential resistance genes and/or mutations in genes that are important for resistance. The machine learning system may be implemented using a feature-wise linear modulation (FiLM) generator deep learning technique to generate multiple layers and blocks that include certain optimization parameters, as discussed in greater detail below. The system disclosed herein uses a FiLM machine learning system. Additionally or alternatively, other modeling systems may be included, such as a conditional batch normalization, gated layers, cross-modal fusion, and/or attention layers.

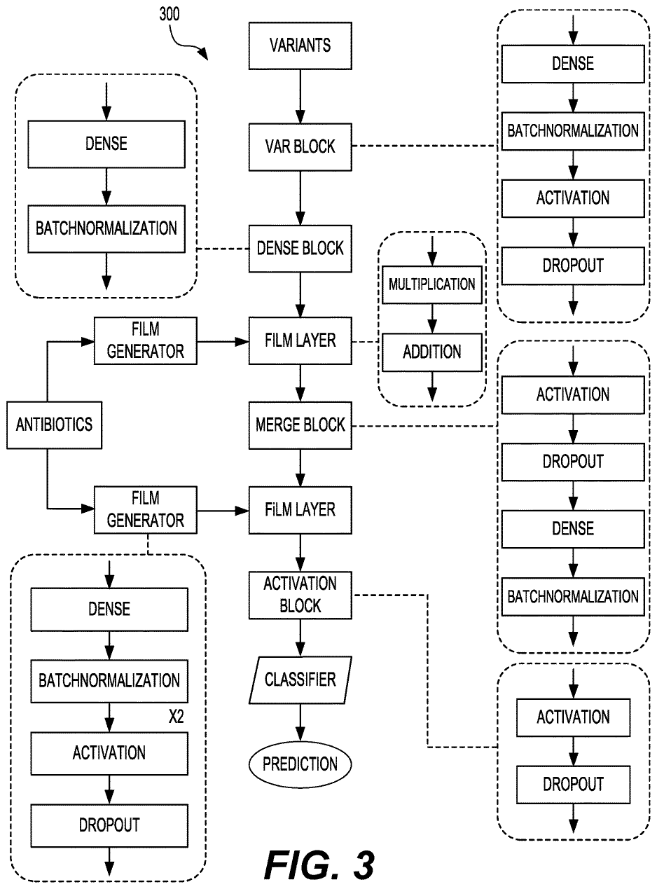

FIG. 3 is a block diagram illustrating an example machine learning system 300, in accordance with certain aspects of the present disclosure. The machine learning system 300 can form at least a portion of any of the antibiotic resistance prediction system(s) 200 and 500-1600 discussed herein. The machine learning system 300 may be used to implement the antibiotic resistance prediction system 200. The machine learning system 300 may be a FiLM machine learning model. The machine learning system 300 can use a deep learning model to jointly model variants and antibiotics, as shown. The machine learning system 300 includes dense block dimensions. A number of FiLM blocks are optimized using nested cross-validations. The model includes a dense/fully connected layer (e.g., a linear operation on the layer's input vector). For activation, the machine learning system 300 may include a rectified linear unit (ReLU) layer. An activation function may be responsible for transforming a summed weighted input from a node into the activation of the node or output for that input. A ReLU layer may be a piecewise linear function that can output the input directly if it is positive, otherwise, it will output zero.

The machine learning system 300 can also include a batch normalization layer and a dropout layer, as shown. Batch normalization may be used to make training of the model faster and more stable through normalization of the layers' inputs by re-centering and re-scaling. A dropout layer may be used to ignore units (e.g., neurons) during a training phase of certain set of neurons which may be chosen at random. For example, these units may not be considered during a particular forward or backward pass. Dropout can reduce interdependence learning among neurons.

As described, the machine learning system 300 may be trained across more than one species of a bacteria. The machine learning system 300 may be trained using protein variants or amino acid variants, as described. For example, in some aspects, instead of using a DNA sequence, the machine learning system 300 may be trained using translated protein variants.

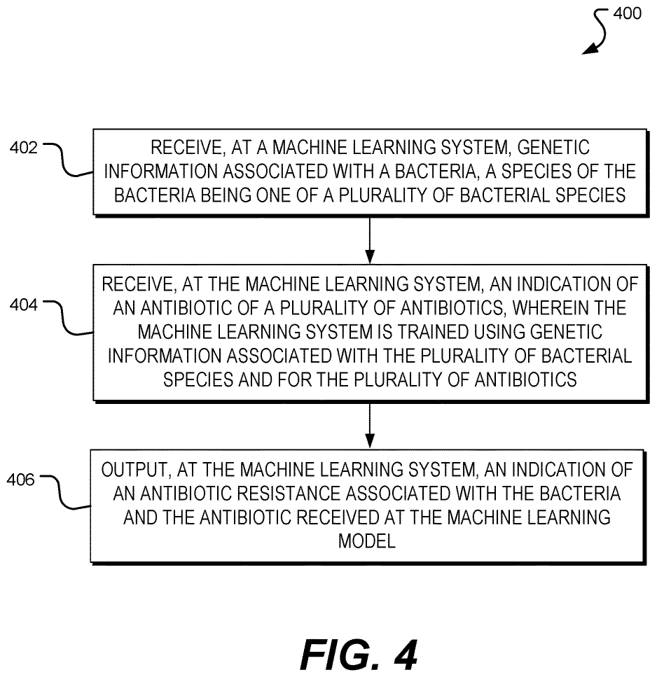

FIG. 4 is a flow diagram illustrating example operations 400 for antibiotic resistance prediction, in accordance with certain aspects of the presently disclosed technology. The operations 400 may be performed, for example, by the computing system 100, the machine learning system 300 and/or any of the antibiotic resistance prediction system(s) 200 and 500-1600.

At block 402, the computing system can receive, at a machine learning system, genetic information associated with a bacteria, a species of the bacteria being one of a plurality of bacterial species. At block 404, the computing system can receive, at the machine learning system, an indication of an antibiotic of a plurality of antibiotics, wherein the machine learning system is trained using genetic information associated with the plurality of bacterial species and for the plurality of antibiotics. At block 406, the computing system can output, at the machine learning system, an indication of an antibiotic resistance associated with the bacteria and the antibiotic received at the machine learning system.

In some aspects, the machine learning system is trained using protein sequences associated with the plurality of bacterial species. In some aspects, the machine learning system comprises a feature-wise linear modulation (FiLM) machine learning system. The machine learning system may jointly model antibiotics and bacterial variants. The machine learning system may include rectified linear activation function (ReLU) layer, a batch normalization layer, and a dropout layer.

These and various other arrangements will be described more fully herein. As will be appreciated by one of skill in the art upon reading the following disclosure, various aspects described herein can be a method, a computer system, or a computer program product. Accordingly, those aspects can take the form of an entirely hardware implementation, an entirely software implementation, or at least one implementation combining software and hardware aspects. Furthermore, such aspects can take the form of a computer program product stored by one or more computer-readable storage media (e.g., non-transitory computer-readable medium) having computer-readable program code, or instructions, included in or on the storage media. Any suitable computer-readable storage media can be utilized, including hard disks, CD-ROMs, optical storage devices, magnetic storage devices, and/or any combination thereof. In addition, various signals representing data or events as described herein can be transferred between a source and a destination in the form of electromagnetic waves traveling through signal-conducting media such as metal wires, optical fibers, and/or wireless transmission media (e.g., air and/or space).

As noted above, implementations of the presently disclosed technology include various steps, which are described in this specification. The steps may be performed by hardware components or may be embodied in machine-executable instructions, which may be used to cause a special-purpose processor programmed with the instructions to perform the steps. Alternatively, the steps may be performed by a combination of hardware, software and/or firmware.

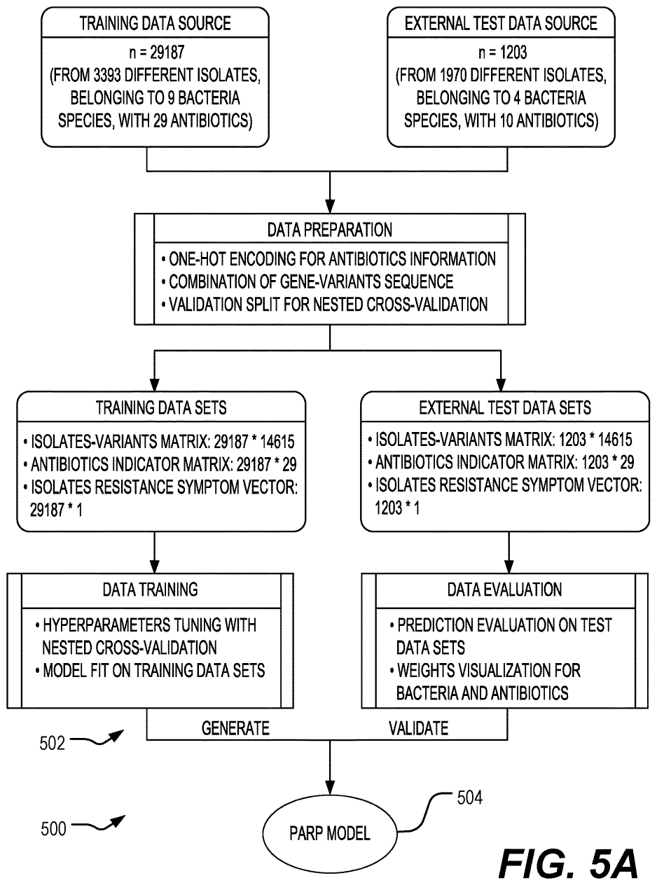

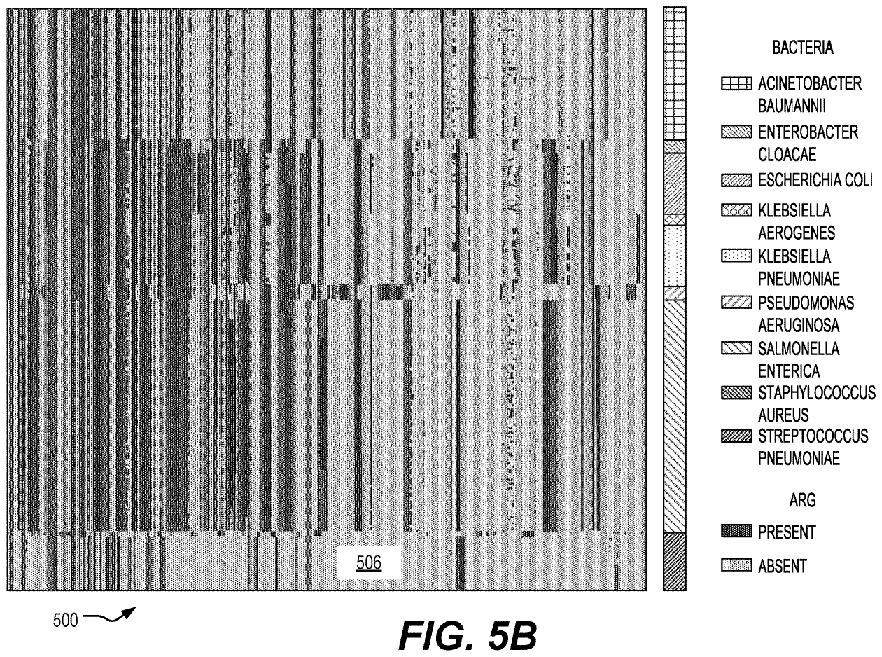

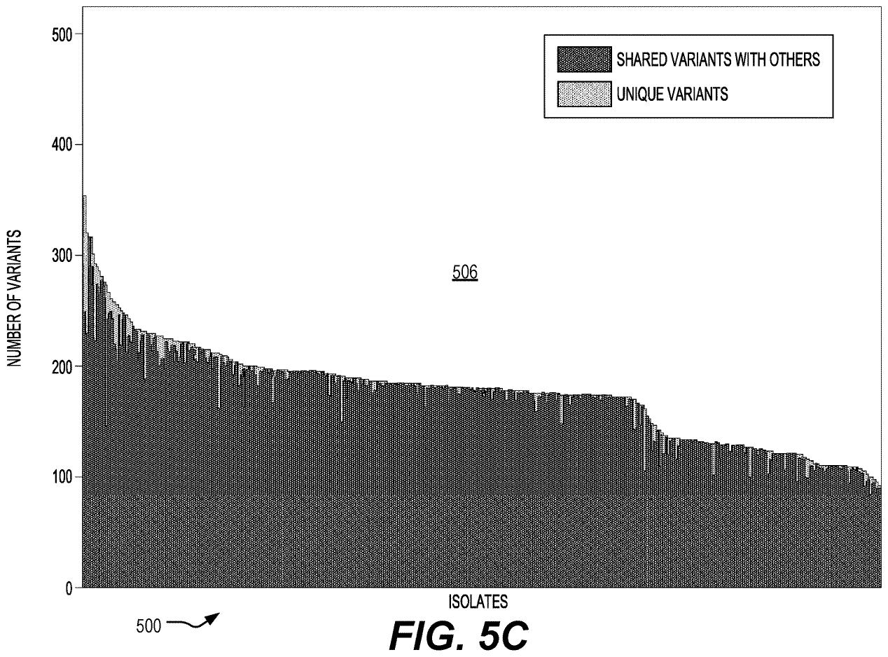

FIGS. 5A-5C illustrate an example antibiotic resistance prediction system 500, in accordance with certain aspects of the presently disclosed technology.

In some examples, as depicted in FIG. 5A the antibiotic resistance prediction system 500 can include a workflow 502 for determining genetic features shared across different pathogen species. The antibiotic resistance prediction system 500 can be a pan-antibiotic resistance prediction model, or PARP model 504, for predicting resistance across a wide variety of pathogens, even those that have not previously been analyzed by the PARP model 504.

In some examples, the antibiotic resistance prediction system 500 includes a curated training dataset with paired bacterial genomes and antibiotics resistance phenotypes, which can be used to train the PARP model 504. The PARP model 504 can also include an independent test dataset used to evaluate the model performance. For example, the data sources can include a training data source of publicly available information which can include 3393 different isolates belonging to 9 bacteria species with 29 different antibiotics. The data sources can also include an external test data source including 1970 different isolates belonging to 4 bacteria species with 10 antibiotics. The PARP model 504 can also include a data preparation procedure for converting the training data and/or the test data into usable training data sets and/or tests data sets. The data preparation procedure can include one-hot encoding the antibiotics feature information, a combination for gene variants sequencing, and/or a validation split for nested cross-validation. Accordingly, the antibiotic resistance prediction system 500 can generate a training data set having a first isolates-variants matrix, a first antibiotics indicator matrix, and/or a first isolates resistance symptom vector. Furthermore, the antibiotic resistance prediction system 500 can generate a test data set including a second isolates-variants matrix, a second antibiotics indicator matrix, and/or a second isolates resistance symptom vector. The PARP model 504 can undergo a data training procedure in which hyperparameters are tuned with nested cross-validation, and a model fit assessment is performed on the training dataset. Moreover, the PARP model 504 can undergo a data validation procedure in which a prediction generated by the PARP model 504 is evaluated using the test data sets. The data training procedure can also include generating weights and/or a weights visualization for bacteria and antibiotics. FIG. 5B depicts a heat map 506 including rows representing samples, columns representing antibiotic resistance genes (ARG), and shading representing an existence of the ARGs. FIG. 5C depicts a graph 508 showing the number of variants for the different isolates sorted in descending order. The darker shading bars represent a first number of variants shared with other isolates, while the lighter shading bars represent a second number of variants that are unique for that isolate.

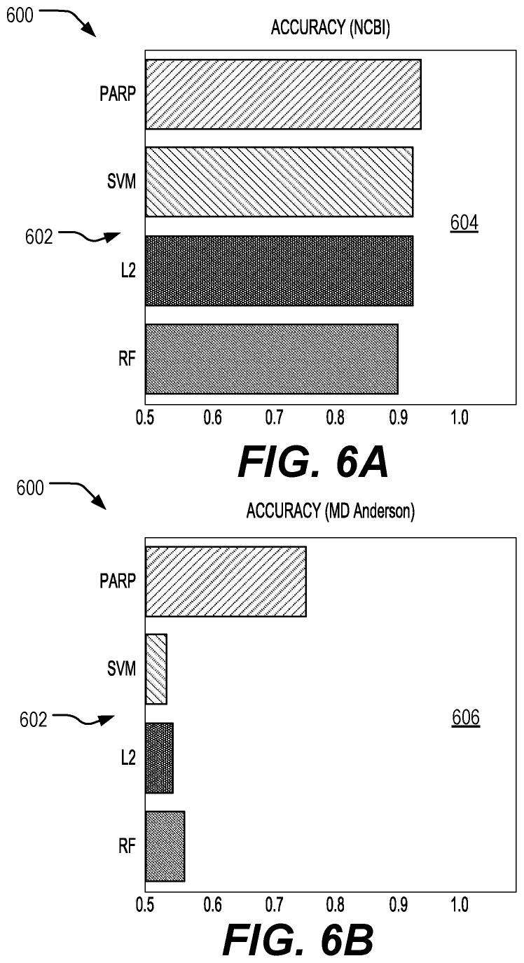

FIGS. 6A and 6B depict an example antibiotic resistance prediction system 600, in accordance with certain aspects of the presently disclosed technology and including a prediction accuracy evaluation 602. For instance, a first prediction accuracy evaluation 604 shown in FIG. 6A can be based on a first test data set (e.g., a National Center for Biotechnology Information (NCBI) data set); and a second prediction accuracy evaluation 606 shown in FIG. 6B can be based on a second test data set (e.g., an MD Anderson Cancer Center data set). According to the prediction accuracy evaluations 602 the PARP model 504 can have improved prediction accuracy over other prediction methods, such as a support vector machine (SVM) model, a logistic regression model with L2 regularization, and/or a random forest (RF) model.

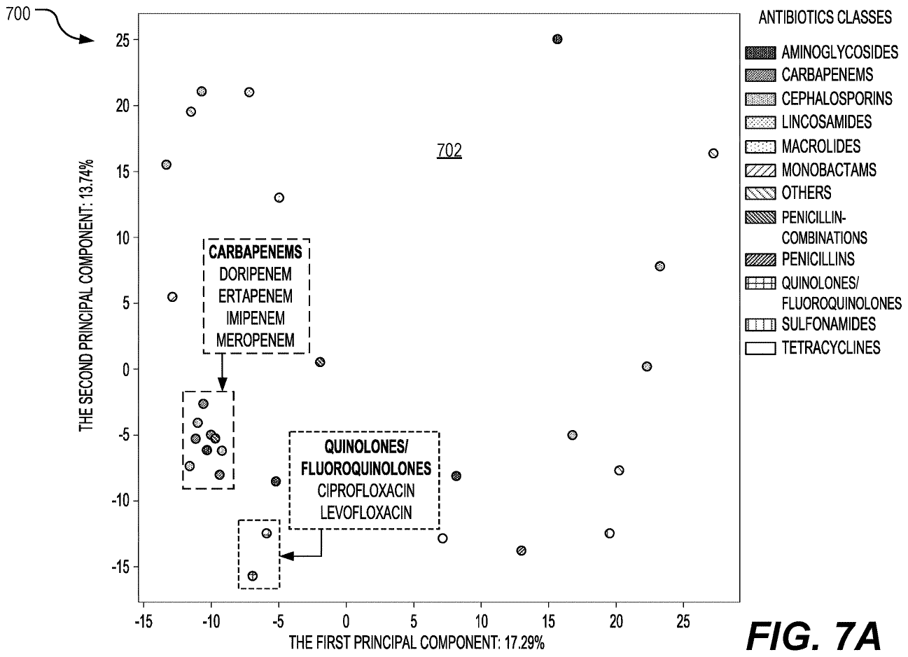

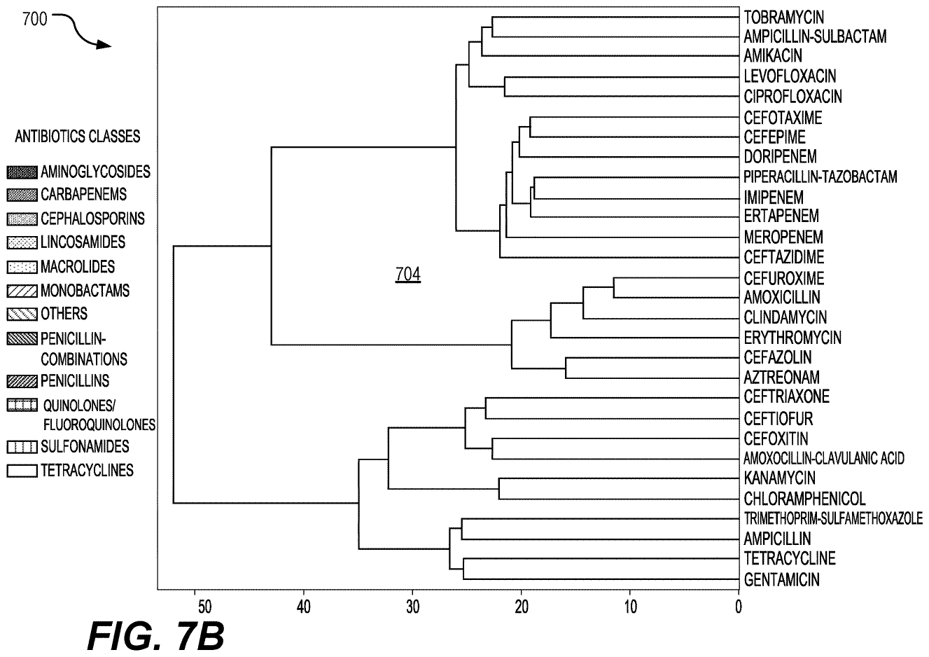

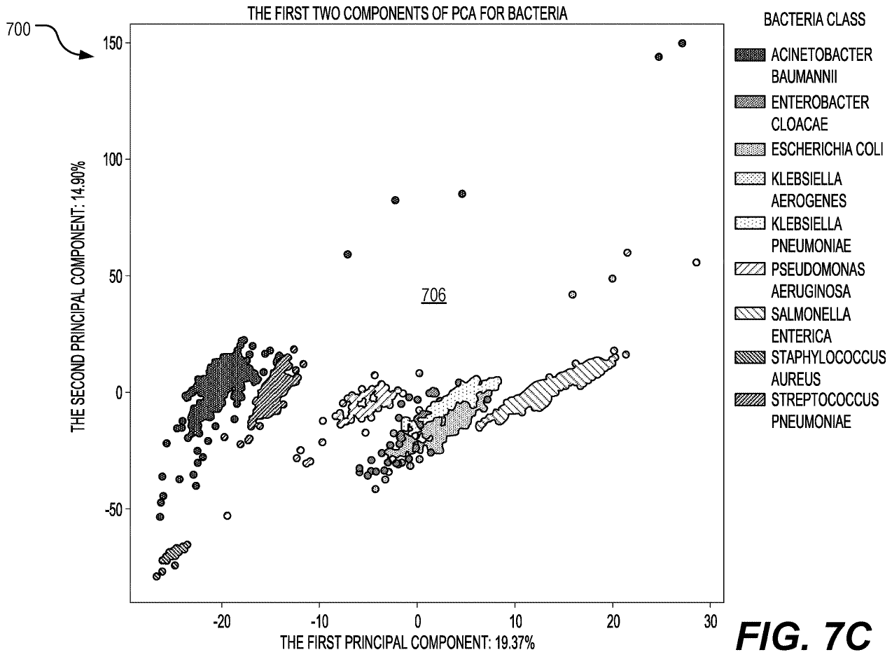

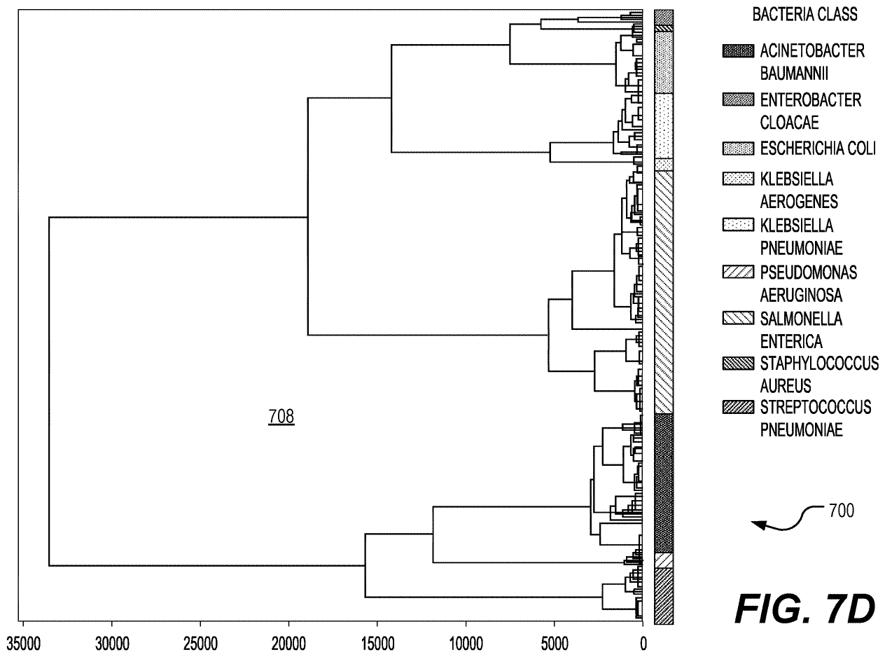

FIGS. 7A-7D depict an example antibiotic resistance prediction system 700 in accordance with the presently disclosed technology including results outputted by the PARP model 504. For example, FIG. 7A depicts a classification graph 702 showing that antibiotics of the same class can be clustered together based on antibiotics embedded on the first principal component and the second principal component. This output can use weights of the four dense layers from the FiLM Generator component of the PARP model 504. For example, Carbapenems can form a first cluster and/or Quinolones/Fluoroquinolones can form a second cluster. FIG. 7B depicts an agglomerative clustering dendrogram 704 of antibiotics, corresponding to the classification graph 702 of FIG. 7A, which can use a Euclidean distance metric and ward linkage criterion, with a cluster threshold being 70% of the maximum linkage value. Furthermore, FIG. 7C depicts a second classification graph 706 for classifying the bacterial isolates. For instance, the second classification graph 706 depicts a third cluster of Acinetobacter baumannii, a fourth cluster of Streptococcus pneumoniae; a fifth cluster of Pseudomonas aeruginosa; a sixth cluster of Klebsiella pneumoniae; a seventh cluster of Escherichia coli; and/or an eight cluster of Salmonella enterica. FIG. 7D depicts an agglomerative clustering dendrogram 708 of bacterial isolates with the Euclidean distance metric and ward linkage criterion, corresponding to the classification graph 706 of FIG. 7. The cluster threshold for the agglomerative clustering dendrogram 708 can be 70% of the maximum linkage.

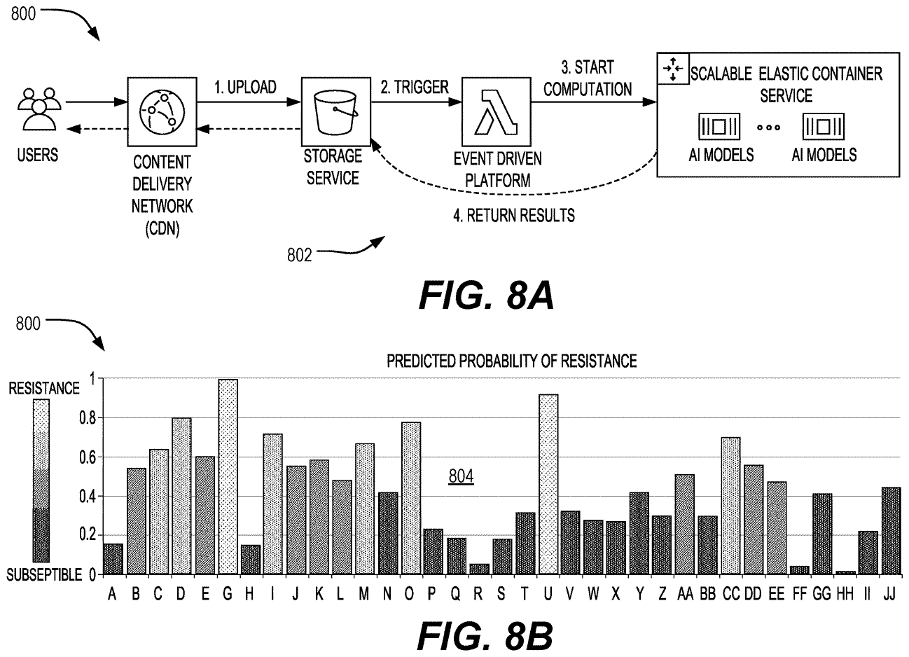

FIG. 8A depicts an example antibiotic resistance prediction system 800 in accordance with the presently disclosed technology including a cloud-based deployment 802. The cloud-based deployment 802 can include a web service portal such as a content delivery network (CDN) accelerated website (e.g., Cloudfront) which a user can access to upload the data of the PARP model 504. The data can be uploaded to a storage service (e.g., S3), which can trigger an event-driven platform, such as a serverless platform like Lambda. Triggering the event-driven platform can initiate a computation process for a container orchestration service (e.g., Elastic Container Service) where the machine learning models 120 disclosed herein (e.g., the PARP model 504) can be executed. Prediction results of the PARP model 504 can be sent from the container orchestration service back to the storage service for browsing, viewing, downloading, or so forth.

FIG. 8B depicts an example antibiotic resistance prediction system 800 in accordance with the presently disclosed technology showing prediction output results 804 of the PARP model 504 (e.g., via the cloud-based deployment 802). These prediction results can correspond to a plurality of different uploaded genomes of different pathogens represented by the x-axis. Lower y-values can correspond to susceptibility to antibiotics and higher y-values can correspond to resistance to antibiotics. By way of example, the prediction output results 804 of the PARP model 504 can be based on a plurality of different antibiotics (e.g., between 10 and 30 antibiotics, or more than 30 antibiotics). The cloud-based deployment 802 can form a decentralized diagnostic test where any remote device can upload any genome sequence via the infrastructure of the cloud-based deployment 802, and the prediction results can be generated and provided to the remote device. In other words, the cloud-based deployment 802 of the PARP model 504 can provide a scalable antibiotic resistance prediction platform over a wide area network (WAN), such as the internet.

In some examples, the prediction output results 804 depicted in FIG. 8B, or throughout this disclosure, can be presented on one or more graphical user interfaces (GUI)s of one or more user devices. For instance, a computing device associated with a clinic, hospital, laboratory, or so forth can receive the output results and/or present the output results at its GUI. In some scenarios, the GUI presenting the prediction output results 804 can be a same GUI that provided an upload of the genome sequence for analysis by the cloud-based deployment 802, or the device presenting the output results can be a different device than that which provided the genome sequence.

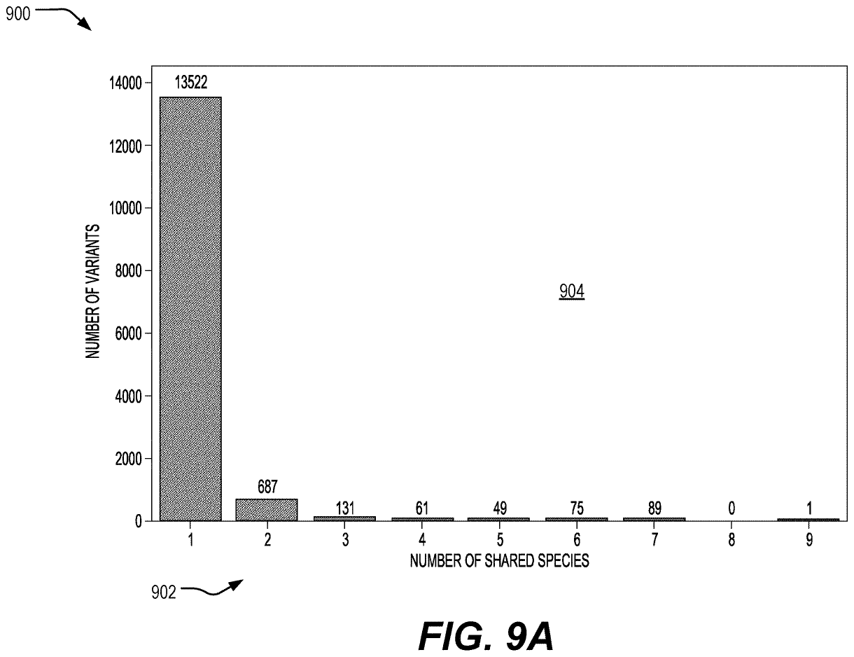

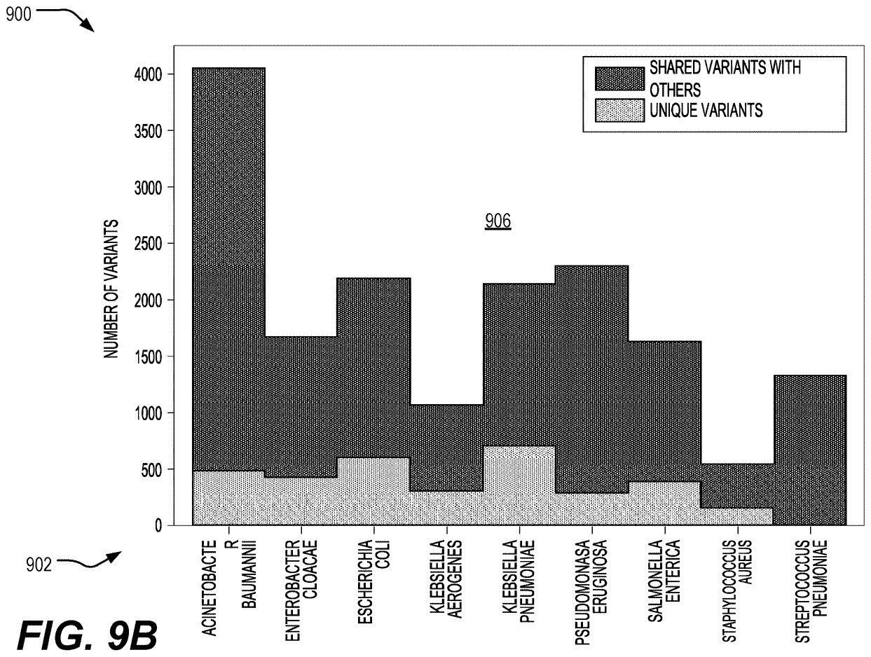

FIGS. 9A and 9B depict example antibiotic resistance prediction systems 900 in accordance with the presently disclosed technology including one or more bar graphs 902 representing unique and/or shared variant data. For instance, a first bar graph 904 shown in FIG. 9A represents the number of antibiotic resistant genes shared among species, as determined by the PARP model 504. A second bar graph 906 shown in FIG. 9B represents shared and unique variants of isolates from the different pathogen species as determined in the training data set. The unique variants, represented by the lighter shaded, bars, are carried only by that particular specie represented on the x-axis. The darker shade bars represent shared variants which are carried by at least two species.

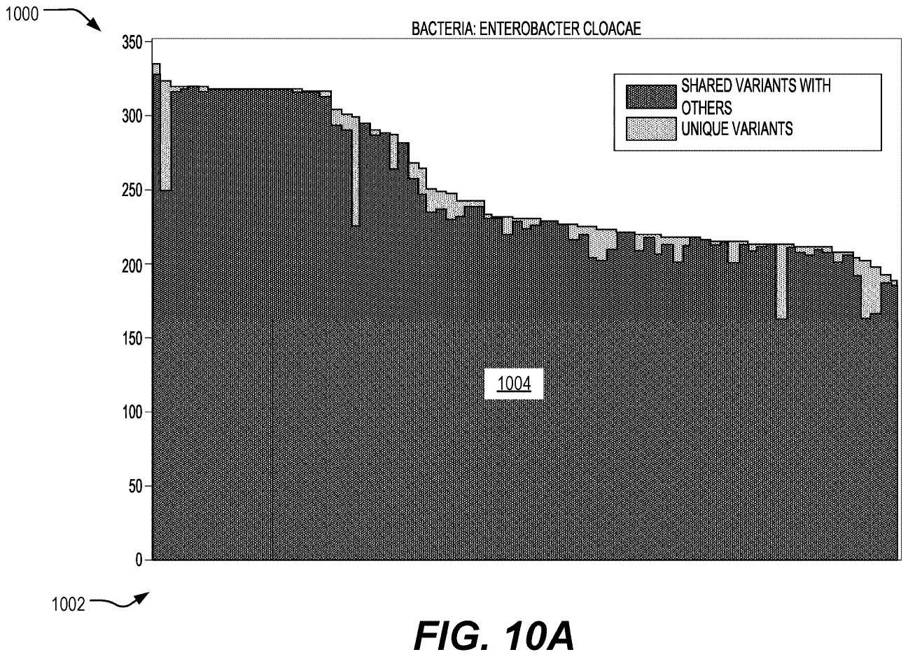

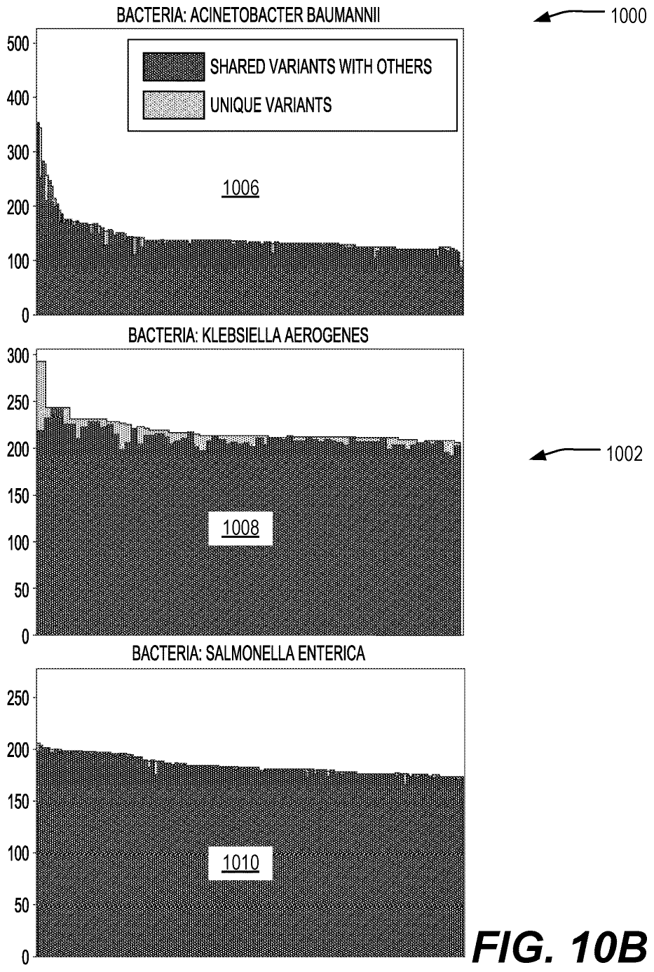

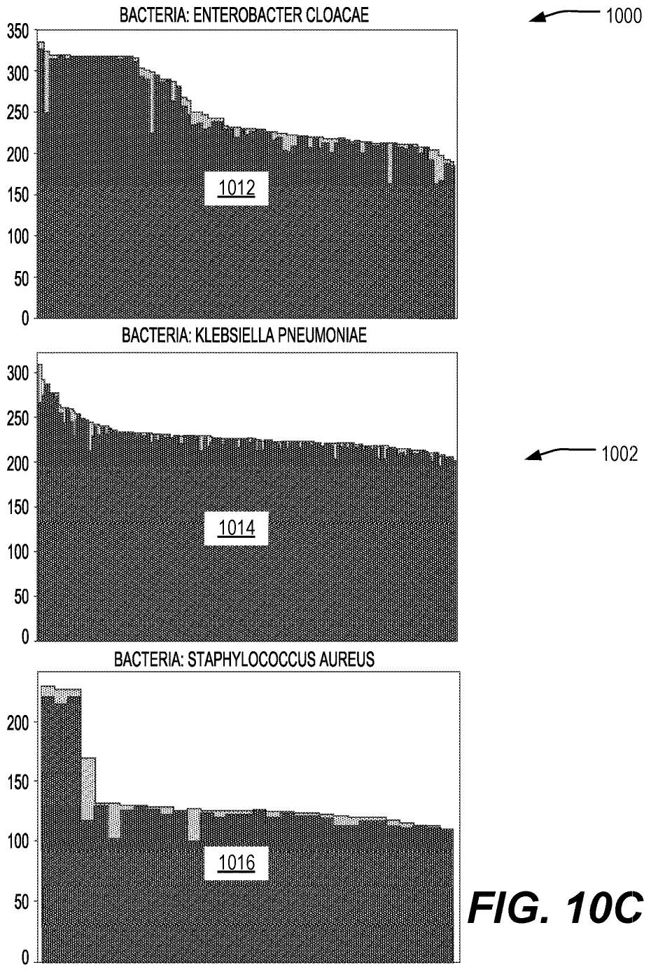

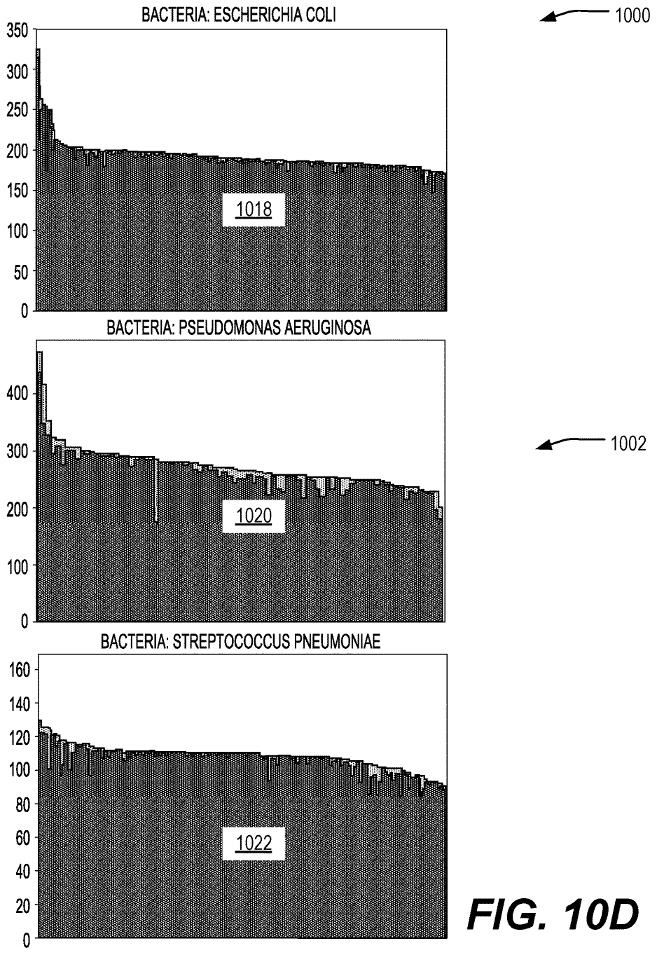

FIGS. 10A-10D depict an antibiotic resistance prediction system 1000 in accordance with the presently disclosed technology including one or more bar graphs 1002 representing unique and/or shared variant data, which can be determined by the PARP model 504. The one or more bar graphs 1002 of FIGS. 10A-10D can represent shared and/or unique variants for a particular pathogen species. The x-axis can represent the isolates, the lighter shade y-value can represent the number of unique variants for that isolate, and the darker shade y-value can represent a number of shared variants for that isolate. For instance, FIG. 10A depicts a first bar graph 1004 representing shared and unique variants by isolate for Enterobacter cloacae. FIG. 10B depicts a second bar graph 1006 representing shared and unique variants by isolate for Acinetobacter baumannii; a third bar graph 1008 representing shared and unique variants by isolate for Klebsiella aerogenes; and a fourth bar graph 1010 representing shared and unique variants by isolate for Salmonella enterica. Furthermore, FIG. 10C depicts a fifth bar graph 1012 representing shared and unique variants by isolate for Enterobacter cloacae; a sixth bar graph 1014 representing shared and unique variants by isolate Klebsiella pneumoniae; and a seventh bar graph 1016 representing shared and unique variants by isolate for Staphylococcus aureus. Additionally, FIG. 10D depicts an eighth bar graph 1018 representing shared and unique variants by isolate for Escherichia coli; a ninth bar graph 1020 representing shared and unique variants by isolate for Pseudomonas aeruginosa; and a tenth bar graph 1022 representing shared and unique variants by isolate for Streptococcus pneumoniae.

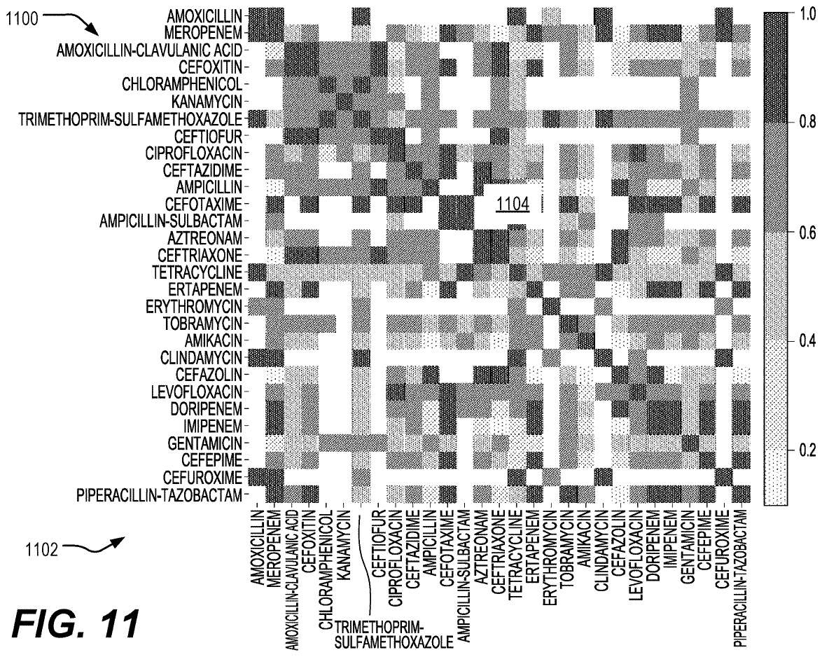

FIG. 11 depicts an antibiotic resistance prediction system 1100 in accordance with the presently disclosed technology including an output of an antibiotic assessment engine 1102, which can form a part of the PARP model 504. In some scenarios, the PARP model 504 can include a two-stage approach including a first stage in which the pathogen genomes are analyzed to determine commonalities and differences which may impact their antibody resistance. The second stage can include an analysis of the antibiotics themselves, using the antibiotic assessment engine 1102, to determine commonalities and differences among the antibiotics with respect to their phenotypes which may impact whether a pathogen is susceptible or resistant to the antibiotic.

For instance, the antibiotic assessment engine 1102 can generate antibiotics susceptibility data represented by a paired-antibiotics susceptibility heat map 1104. For each pair of antibiotics in the paired-antibiotics susceptibility heat map 1104, a square represents the proportion of isolates with the identical phenotype which were tested on both the x-axis antibiotic and the y-axis antibiotic. Blank squares indicate that no isolates were tested on that particular antibiotic combination. The paired-antibiotics susceptibility heat map 1104 can indicate how different classes of antibiotics target different pathways. The PARP model 504 can integrate the results of the paired-antibiotics susceptibility heat map 1104 into its determination of antibiotic resistance for different variants via extrapolation of the identical phenotypes. In this way, the PARP model 504 can make a prediction for a particular antibiotic, even if that antibiotic has not been specifically tested, by recognizing its similarities to other antibiotics that have been tested.

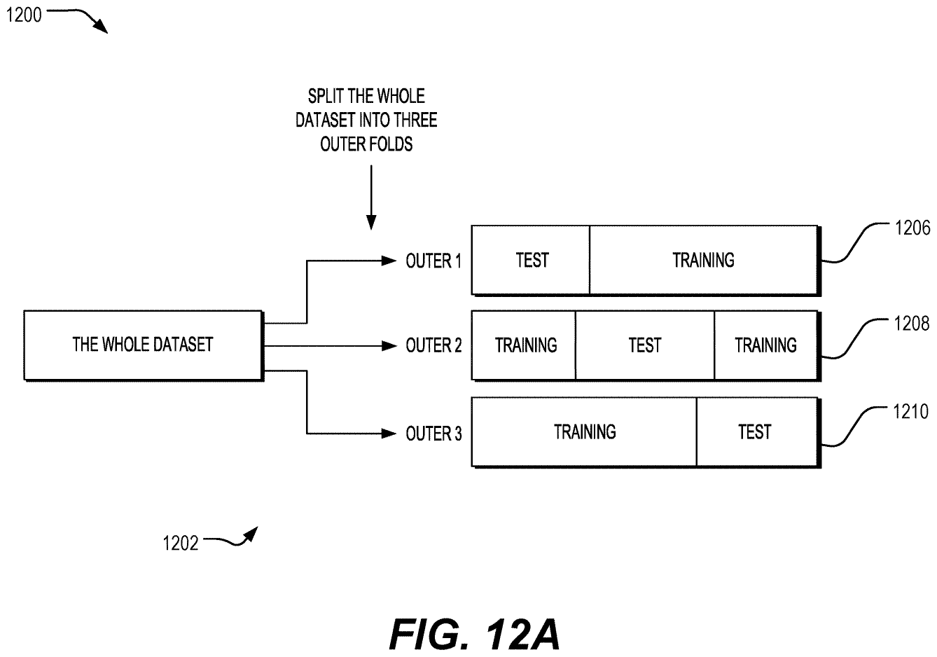

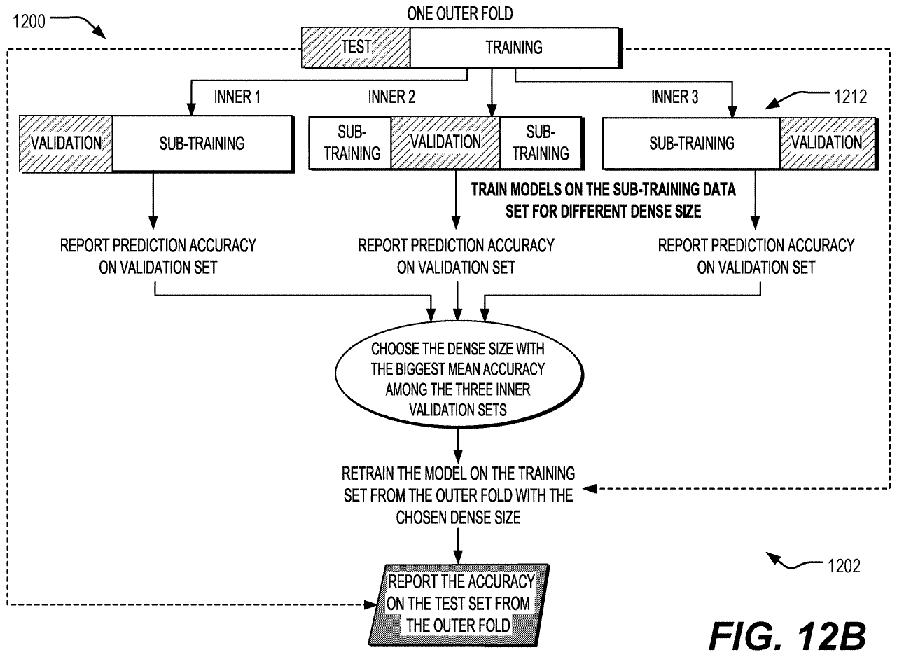

FIGS. 12A and 12B depict an example antibiotic resistance prediction system 1200 in accordance with the presently disclosed technology including a nested cross-validation procedure 1202. For instance, FIG. 12A depicts an outer loop 1204 of the nested cross-validation procedure 1202 in which the training data set can be split into three outer folders that take a third of the data as test data and two thirds of the data as training data. The training data set can be shuffled before this splitting to ensure that the selected datasets are representative of the overall data. A first outer folder 1206 can use the first third of the data set as test data and the latter two thirds of the data set as training data. A second outer folder 1208 can use the first third and the last third of the data set as training data and the middle third as test data. A third outer folder 1210 can use the first two thirds of the data set as the training data and the latter third of the data set as the test data. In some instances, results of the first outer folder 1206, the second outer folder 1208, and/or the third outer folder 1210 can be combined together. FIG. 12B depicts an inner loop 1212 of the nested cross-validation procedure 1202. The inner loop 1212 depicted in FIG. 12B corresponds to the first outer folder 1206, although a similar or identical inner loop 1212 can be used for the second outer folder 1208 and/or the third outer folder 1210. The inner loop 1212 can include splitting the outer fold into three inner folds which take 20% of the data set as the validation data set. These validation data sets can be used for choosing hyperparameters and the prediction metrics on the tests subset can be used as the training metrics. The three inner folds can report a prediction accuracy on the validation set, and the nested cross-validation procedure 1202 can choose the dense size with the largest mean accuracy among the three inner validation sets. Then the model can be retrained on the training set from the outer fold with the chosen dense size. Finally, the inner loop 1212 can report the accuracy on the test set from the outer fold. The various operations of the nested cross-validation procedure 1202 disclosed herein can, in some instances, reduce bias in the PARP model 504.

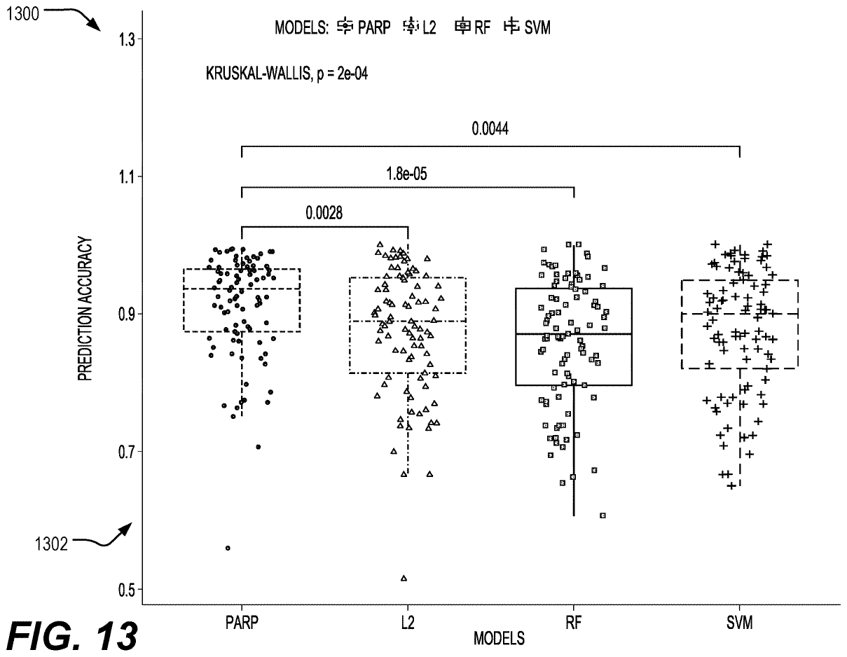

FIG. 13 depicts an example antibiotic resistance prediction system 1300 in accordance with the presently disclosed technology including a prediction output comparison 1302. For instance, the prediction output comparison 1302 can include a box plot of prediction accuracies on the training set using the PARP model 504, which can be compared to other prediction models. For instance, the prediction output comparison 1302 can generate a prediction comparison between the PARP model 504 and the logistic regression model with L2 regularization, the RF model, and/or the SVM model. In some examples, pairwise p-values between the PARP model 504 and the other models can be from a Wilcoxon signed-rank test, and/or a Kruskal-Wallis test, which can be used to compare the results among the four models. In some scenarios, the prediction output comparison 1302 can indicate a higher degree of accuracy (e.g., via a tighter box cluster) for the PARP model 504 as compared to the other models.

In some examples, the overall accuracy of the PARP model 504 on the training data can be 94.8%, while 64 out of 93 bacteria-antibiotics pairs can have accuracies above 90% and 83 pairs have accuracies above 80%. When compared with existing machine methods, the PARP model 504 can have the best performance (e.g., PARP 94.8%, Elastic net L2 93.2%, RF 91.1%, and SVM 93.2%).

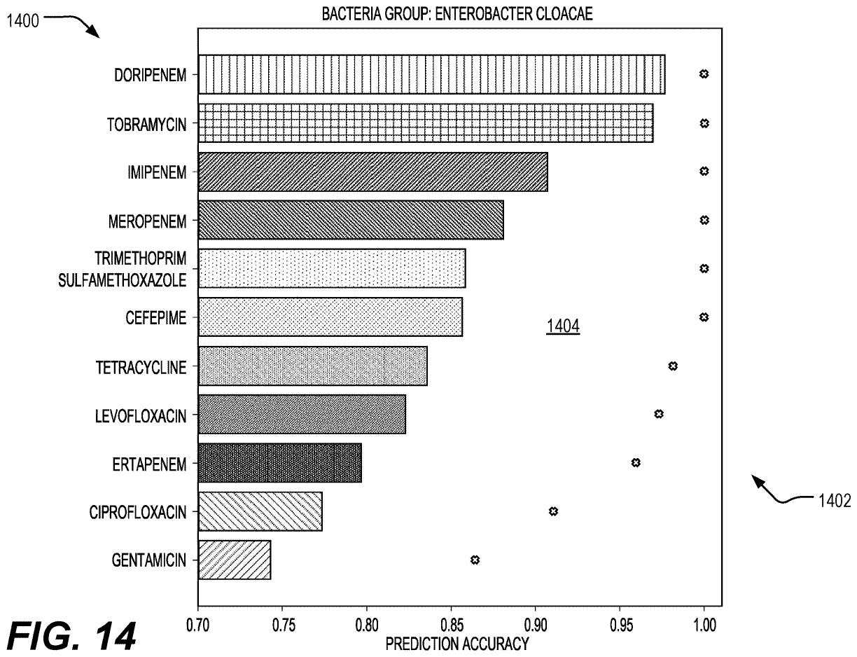

FIG. 14 depicts an example antibiotic resistance prediction system 1400 in accordance with the presently disclosed technology including a prediction accuracy assessment 1402 for unseen bacterial and antibiotic combinations. The prediction accuracy assessment 1402 can predict accuracies for different bacterium-antibiotic pairs based on the PARP model 504 trained on the subset of the data set, as discussed above. The prediction accuracy assessment 1402 can include a bar graph 1404 corresponding to a particular bacterium group, such as the Enterobacter cloacae group. A cross symbol at the different bar graphs represents the prediction training accuracy of isolates from this pair on the original PARP model 504. The threshold can be set as 0.5. Although the Enterobacter cloacae group assessment is depicted in FIG. 14, it is to be understood that a plurality of prediction accuracy assessments 1402 generating a plurality of bar graphs 1404 can be used for a plurality of different bacterium groups, such as Acinetobacter baumannii, Escherichia coli, Klebsiella aerogenes, Klebsiella pneumoniae, Pseudomonas aeruginosa, Salmonella enterica, Staphylococcus aureus, and/or Streptococcus pneumoniae.

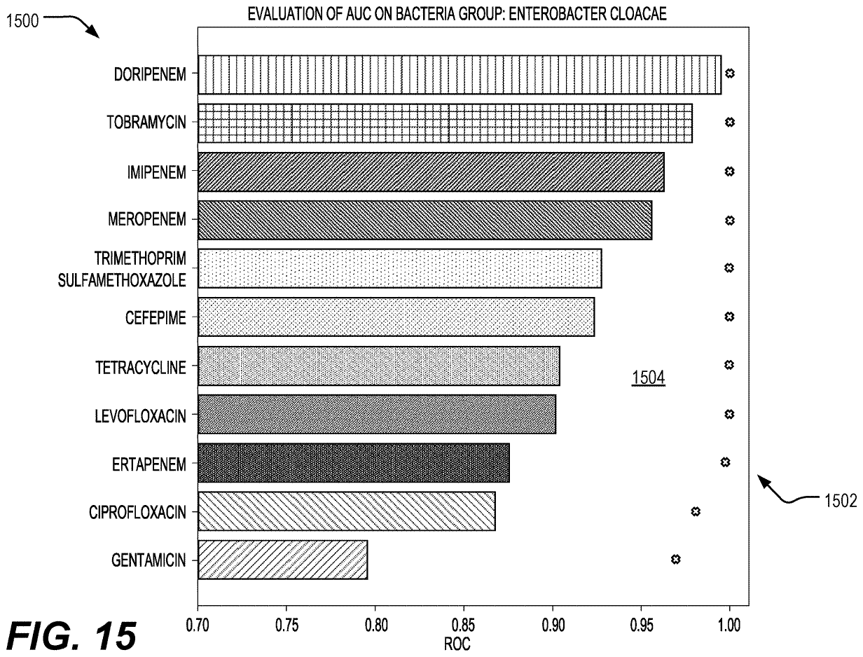

FIG. 15 depicts an example antibiotic resistance prediction system 1500 in accordance with the presently disclosed technology including a prediction area under a receiver operating characteristic curve (AUROC) 1502 for unseen bacterial and antibiotic combinations. The prediction AUROC 1502 can predict accuracy for the different bacterium-antibiotic pairs based on the PARP model 504 trained on the subset of the data set, which excludes isolates from the pairs. The prediction AUROC 1502 can include a bar graph 1504 corresponding to a particular bacterium group, such as Enterobacter cloacae. A cross symbol at the different bar graphs represents the prediction training AUROC of isolates from this pair on the original PARP model 504. Although the Enterobacter cloacae group assessment is depicted in FIG. 15, it is to be understood that a plurality of AUROC 1502 generating a plurality of bar graphs 1504 can be used for a plurality of different bacterium groups, such as Acinetobacter baumannii, Escherichia coli, Klebsiella aerogenes, Klebsiella pneumoniae, Pseudomonas aeruginosa, Salmonella enterica, Staphylococcus aureus, and/or Streptococcus pneumoniae.

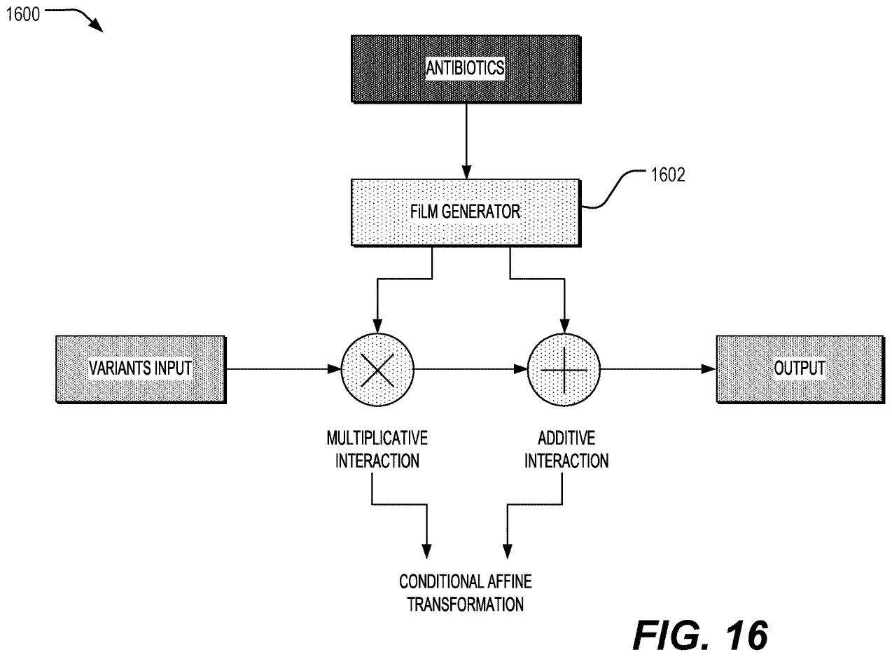

FIG. 16 depicts an example antibiotic resistance prediction system 1600 in accordance with the presently disclosed technology including a Feature-wise Linear Modulation (FiLM) generator block 1602. The block 1602 can include the FiLM machine learning model discussed above regarding FIG. 3. In some scenarios, the antibiotic resistance prediction system 1600 includes the structure of conditional affine transformation. After the transformation from the FiLM generator block 1602, the information from the antibiotic one-hot matrix can be merged into the deep learning model using two interactions, such as a multiplicative interaction and an additive interaction.

In some examples, the antibiotic resistance prediction system(s) 200 and 500-1600 depicted in FIGS. 1-16 can address the emerging public health threat of Antibiotic Resistance (AR). Additional details of the antibiotic resistance prediction system(s) 200 and 500-1600 are provided below.

The antibiotic resistance prediction system(s) 200 and 500-1600 can provide an advancement of whole-genome bacterial sequencing technologies and machine learning by providing in silico antimicrobial resistance prediction results in a timely and accurate fashion. The bioinformatic methods disclosed herein can profile bacterial sequences for machine learning features. These prediction models generated by the antibiotic resistance prediction system(s) 200 and 500-1600 can use both genomic features and antimicrobial susceptibility test (AST) data to facilitate AR prediction. The antibiotic resistance prediction system(s) 200 and 500-1600 disclosed herein can address issues related to the limited data availability of paired bacterial genomes and their AST phenotypes which can create challenges for building accurate prediction models.

For example, the antibiotic resistance prediction system(s) 200 and 500-1600 can include a deep learning model, such as the machine learning model 120, to reveal the relationship between antibiotic resistance genes (ARGs) and a wide range of antibiotics. The antibiotic resistance prediction system(s) 200 and 500-1600 can use ortholog gene variants as input machine learning features to identify their links to antibiotic resistance. The approach disclosed herein can provide at least two advantages. Ortholog-based features can have identifiable and/or explainable relationships between variants and antibiotics, which may not be the case in other approaches that merely check the presence of ARGs or the counts of short DNA fragments (e.g., k-mers). Also, some other models rely on the availability of paired bacterial genome features and their phenotypes (e.g., resistance or susceptibility phenotype of each antibiotic), thus the prediction task can be difficult for these other models when applied to under-represented bacteria constrained by a lack of data availability.

In some examples, the antibiotic resistance prediction system(s) 200 and 500-1600 can include a machine learning framework to study the protein variants across at least nine bacterial species (e.g., or more or less bacterial species) and/or at least 29 antibiotics (e.g., or more or less antibiotics). The antibiotic resistance prediction system(s) 200 and 500-1600 can identify similar protein variants with similar antibiotic functions across different bacterial species or antibiotics classes. Thus, the developed deep learning prediction model(s) of antibiotic resistance prediction system(s) 200 and 500-1600 can be suitable to predict antibiotic resistance across a wide range of bacterial species. The antibiotic resistance prediction system(s) 200 and 500-1600 can predict resistance even for bacterial species for which small numbers of genomes are available.

In some examples, a workflow of the antibiotic resistance prediction system(s) 200 and 500-1600, such as the workflow 502 discussed above regarding FIGS. 5A-5C, can start with the curation of paired bacterial genomes and their AR phenotypes. After quality control procedures, a large dataset of 3,393 isolates with paired AST results can be curated for the antibiotic resistance prediction system(s) 200 and 500-1600. Next, the antibiotic resistance prediction system(s) 200 and 500-1600 can determine the sharing of genetic features related to antibiotic resistance across species followed by blending of shared genetic and antibiotic features. The PARP model 504 can be optimized and its performance unbiasedly evaluated through the nested cross-validation procedure 1202 discussed above regarding FIGS. 12A and 12B. Compared with other prediction models, the PARP model 504 can have a high accuracy (e.g., 94.8%). By interrogating model parameters, the antibiotic resistance prediction system(s) 200 and 500-1600 can determine that the PARP model 504 is explainable, as representing an intrinsic relationship between AR and bacterial taxonomy. Moreover, the antibiotic resistance prediction system(s) 200 and 500-1600 can validate the performance of the PARP model 504 using an independent dataset (e.g., including 197 isolates of 4 bacterial species).

In some examples, the antibiotic resistance prediction system(s) 200 and 500-1600 can determine antibiotic resistance features which are shared across species.

For example, a large collection of paired bacterial genomes and AST data can be curated for the antibiotic resistance prediction system(s) 200 and 500-1600 by accessing an NCBI antibiogram database and/or an NCBI Short Read Archive. As a quality control procedure, the antibiotic resistance prediction system(s) 200 and 500-1600 can exclude bacteria genomes with ambiguous species identity and/or poor sequencing coverage and can categorize minimum inhibitory concentration (MIC) test results using Clinical & Laboratory Standards Institute (CLSI) breakpoints. Additionally, a final cohort can include 9 bacteria species, 3,393 isolates, totaling 29,187 binary AR test results.

In some scenarios, a next step can include using the antibiotic resistance prediction system(s) 200 and 500-1600 to derive genetic features related to AR from some or all species. The information between ARG and antibiotics can be learned across multiple species and antibiotics. This is in contrast to other machine learning models where each model is suitable for one combination of bacterial species and antibiotics and can be limited by the sample size of available paired bacterial genome features and the resistance phenotypes. To develop a unified model where all bacteria genome features and their pan-antibiotic resistance profiles can be studied, the antibiotic resistance prediction system(s) 200 and 500-1600 can determine amino-acid changes occurring in orthologous genes. For the different bacterial genomes, the antibiotic resistance prediction system(s) 200 and 500-1600 can characterize these amino-acid changes using specific bioinformatics approaches, as discussed herein. The antibiotic resistance prediction system(s) 200 and 500-1600 can determine that 406 variants are shared across multiple species and/or 174.7(±42.6) variants are shared across the bacterial isolates. The antibiotic resistance prediction system(s) 200 and 500-1600 can also determine that, among 402 AR genes, 250 genes are shared with more than two species. Moreover, the antibiotic resistance prediction system(s) 200 and 500-1600 can determine that, on average, any particular bacteria species can carry 82.3%-100% of the genes that were also observed in other species. Additionally, the antibiotic resistance prediction system(s) 200 and 500-1600 can determine that bacterial isolates can exhibit frequent resistance across antibiotics in similar classes. For example, the antibiotic resistance prediction system(s) 200 and 500-1600 can determine that AST outcomes are similar for doripenem and imipenem. The antibiotic resistance prediction system(s) 200 and 500-1600 can use the extensive sharing of genetic features and concordance among antibiotic phenotypes to provide a unified predictive machine learning framework for broad range predictions, thus forming the PARP model 504.

In some examples, the antibiotic resistance prediction system(s) 200 and 500-1600 can include the PARP model 504 with a deep learning model to predict antibiotics resistance across multiple combinations of bacterial species and antibiotics.

For example, the PARP model 504 can use paired genetic features and antibiotics as inputs and AST outcomes as outputs. For instance, the inputs of the PARP model 504 can include at least one of protein-level ortholog gene features (e.g., denoted by the Kyoto Encyclopedia of Genes and Genomes (KEGG)), ortholog genes name, ortholog gene variants, and/or AST data. One-hot encoding can be used for a set of antibiotics (e.g., 29 antibiotics) included in the dataset. The design of the model architecture can be optimized by the antibiotic resistance prediction system(s) 200 and 500-1600 to embed and/or blend information from both genetic and antibiotic features through nested cross validation (e.g., the nested cross-validation procedure 1202 of FIG. 12). An optimal model of the PARP model 504 can be determined with one dense block, two FiLM generators, and/or a dense size which can be 1024.

In some examples, the PARP model 504 can predict resistance for both pathogens in the training set as well as those not in the training set by determining the frequently shared genetic features that the pathogens contain. To assess performance of the PARP model 504 on unseen bacteria-antibiotic combinations, the antibiotic resistance prediction system(s) 200 and 500-1600 can perform a leave-one-combination-out (LOCO) procedure in which for a given bacteria-antibiotic combination, the PARP model 504 can be trained with the samples not in this combination and predict resistance for those in this combination. This LOCO procedure can be repeated for a plurality of bacteria-antibiotic combination (e.g., for all 93 combinations).

In some examples, the antibiotic resistance prediction system(s) 200 and 500-1600 can perform external validation using independent datasets.

For example, the performance of the PARP model 504 can be evaluated using an independent test dataset independently collected at MD Anderson (e.g., as discussed above regarding FIG. 6B). The test can use, for example, 197 unique isolates from 4 different bacterial species: Enterobacter cloacae (N=13), Escherichia coli (N=31), Klebsiella pneumoniae (N=24), and Pseudomonas aeruginosa (N=129). These isolates can be tested on 10 various antibiotics and hence, 1,203 samples from 21 pathogen-drug pairs can finally be included in the test dataset. The PARP model 504 trained using all 3,393 bacterial isolates from the NCBI Antibiogram dataset can be evaluated. The PARP model 504 can have the highest accuracy for the Escherichia coli and meropenem combination (accuracy=93.55%). The overall accuracy of the PARP model 504 can be 76.56%, which can be better than other approaches (e.g., SVM=53.11%, Elastic net L2=54.42%, RF=56.28%)).

Next, the PARP model 504 can be evaluated to determine whether the PARP model 504 can predict novel bacteria-antibiotic combinations not seen in the training datasets. For example, the Escherichia coli and meropenem combination can be excluded from an NCBI Antibiogram dataset. The PARP model 504 can be retrained using the reduced dataset and can predict resistance on the MD Anderson dataset with the prediction accuracy of 93.55%.

In some scenarios, the antibiotic resistance prediction system(s) 200 and 500-1600 can determine explainable genetic features via embeddings.

For example, after a final prediction model of the PARP model 504 is developed, the antibiotic resistance prediction system(s) 200 and 500-1600 can explore whether network parameters from the trained model reflect the hidden relationships between genetic features and antibiotics. A principal component analysis (PCA) and/or a hierarchical clustering analysis (HCA) can be performed based on the feature maps inside the dense layers for unsupervised classification for bacteria and antibiotics, respectively. The PARP model 504 can represent relationships among different antibiotics or bacterial species without prior information about these relationships being provided. FIG. 7A shows the antibiotics embedded on the first 2 principal components. Although the first two dimensions can, in some scenarios, only explain 31.01% of total variance, the antibiotic resistance prediction system(s) 200 and 500-1600 can determine that the hidden representation of antibiotic features tend to be clustered by their classes, such as carbapenems or quinolones, as highlighted in the dashed boxes of FIG. 7A. This can indicate that the PARP model 504 automatically captures the features shared within the same antibiotics class. FIG. 7B further shows the antibiotics that are clustered by their class. In addition, FIG. 7C includes a visualization of the 9 bacteria involved in the training dataset in terms of the first two principal components. Samples can be clustered with others belonging to the same bacterium. As such, the PARP model 504 can learn information to distinguish different bacteria.

Furthermore, techniques for integrating the bacterial genetic features and AST data can be investigated by the antibiotic resistance prediction system(s) 200 and 500-1600. A model agnostic method can be used by artificially inducing different genetic features and quantifying the change of the predicted resistance. A higher value can reflect that a variant is more important to inducing resistance. For instance, the antibiotic resistance prediction system(s) 200 and 500-1600 can determine that the K18768 variant on blaKPC (beta-lactamase class A KPC) contributes the largest resistance for doripenem, imipenem, and meropenem. Similarly, the K19096 mutation on aph3-I (aminoglycoside 3′-phosphotransferase I), a gene that modifies aminoglycoside antibiotics (e.g., amikacin), can contribute to its largest resistance.

In some scenarios, the antibiotic resistance prediction system(s) 200 and 500-1600 can facilitate the use of the PARP model 504 with the broader research community by providing a website for the developed PARP model 504 (e.g., as depicted above regarding FIG. 8A). The website can provide a portal for users to upload genome sequences and/or obtain an in-silico predicted resistance profile within minutes. Users can receive a report including the probability of resistance of a plurality of antibiotics, such as 35 antibiotics, as depicted in FIG. 8B. Moreover, this cloud-based deployment 802 can enhance scalability, affordability, and availability of the PARP model 504 in at least three ways. First, the software pipeline can be packaged to extract ortholog gene features and to compute the resistance prediction in a software container so that the cloud-based deployment 802 can scale up and reproducibly perform online analyses concurrently. Second, the website can use a serverless architecture, so computation incurs minimal costs only when the computation occurs. Additionally, the cloud-based deployment 802 can have built-in backup and replication mechanisms so that the server can provide uninterrupted service for worldwide researchers.

In some examples, the antibiotic resistance prediction system(s) 200 and 500-1600 can use various data acquisition techniques.

For example, the training dataset can be processed based on the NCBI BioSample Antibiograms database. The antibiogram tabular data can be used to verify bacterial isolates and the reported minimum inhibitory concentration (MIC) values can be retained. The corresponding sequencing data can be from the NCBI Sequence Read Archive (SRA). In some scenarios, the PARP model 504 can keep the bacterial isolates which have consistent species from antibiogram and from sequence data analysis, and the resistant and susceptible phenotypes can be based on a CLSI standard.

In the training dataset, 3,393 unique bacterial isolates representing 9 species can be included, such as: Acinetobacter baumannii (772), Enterobacter cloacae (79), Escherichia coli (350), Klebsiella aerogenes (68), Klebsiella pneumoniae (344), Pseudomonas aeruginosa (83), Salmonella enterica (1349), Staphylococcus aureus (31), and Streptococcus pneumoniae (317). Moreover, their resistance phenotypes to 29 different antibiotics can be curated. As such the antibiotic resistance prediction system(s) 200 and 500-1600 can obtain 29,187 paired pathogen-antibiotic samples covering 93 various specie-antibiotic combinations.

In an external dataset from MD Anderson, the antibiotic resistance prediction system(s) 200 and 500-1600 can sequence 197 unique isolates. This dataset can serve as a validation cohort for the developed model, and can include 4 bacteria, such as Enterobacter cloacae (13 isolates), Escherichia coli (31 isolates), Klebsiella pneumoniae (24 isolates), and Pseudomonas aeruginosa (129 isolates). These isolates can have paired antibiotic test phenotypes totaling 1,203 paired pathogen-antibiotics covering 21 species-antibiotic combinations.

In some aspects, the training dataset can comprise a plurality of bacteria species (e.g., two or more), wherein plurality of bacteria are from one or more of genus including, but not limited to, Yersinia, Vibrio, Treponema, Streptococcus, Staphylococcus, Shigella, Salmonella, Rickettsia, Orientia, Pseudomonas, Neisseria, Mycoplasma, Mycobacterium, Listeria, Leptospira, Legionella, Klebsiella, Helicobacter, Haemophilus, Francisella, Escherichia, Ehrlichia, Enterococcus, Coxiella, Corynebacterium, Clostridium, Chlamydia, Chlamydophila, Campylobacter, Burkholderia, Brucella, Borrelia, Bordetella, Bifidobacterium, Bacillus, Proteus, Morganella, Sphingobium, Sphingomonas, Zymomonas, Cupriavidus, or any combination thereof.

In some aspects, the plurality of bacteria species are selected from Achromobacter spp, Acidaminococcus fermentans, Acinetobacter calcoaceticus, Actinomyces spp, Actinomyces viscosus, Actinomyces naeslundii, Aeromonas spp, Aggregatibacter actinomycetemcomitans, Anaerobiospirillum spp, Alcaligenes faecalis, Arachnia propionica, Bacillus spp, Bacteroides spp, Bacteroides gingivalis, Bacteroides fragilis, Bacteroides intermedius, Bacteroides melaninogenicus, Bacteroides pneumosintes, Bacterionema matruchotii, Bifidobacterium spp, Buchnera aphidicola, Butyriviberio fibrosolvens, Boretella pertussis, Campylobacter spp, Campylobacter coli, Campylobacter sputorum, Campylobacter upsaliensis, Capnocytophaga spp, Chlamydophila pneumoniae, Clostridium spp, Citrobacter freundii, Clostridium difficile, Clostridium sordellii, Corynebacterium spp, Eikenella corrodens, Enterobacter cloacae, Enterococcus spp, Enterococcus faecalis, Enterococcus faecium, Escherichia coli, Eubacterium spp, Flavobacterium spp, Fusobacterium spp, Fusobacterium nucleatum, Gordonia Bacterium spp, Haemophilus parainfluenzae, Helicobacter pylori, Haemophilus paraphrophilus, Klebsiella, Lactobacillus spp, Listeria monocytogenes, Leptotrichia buccalis, Methanobrevibacter smithii, Micrococcus flavus, Moraxella catarrhalis, Mycobacteria tuberculosis, Mycobacteria paratuberculosis, Mycoplasma pneumonie Morganella morganii, Mycobacteria spp, Mycoplasma spp, Micrococcus spp, Mycobacterium chelonae, Neisseria spp, Neisseria sicca, Pasteurella multocida, Peptococcus spp, Peptostreptococcus spp, Plesiomonas shigelloides, Porphyromonas gingivalis, Proteus spp, Proteus mirabilis, Proteus vulgaris, Propionibacterium spp, Propionibacterium acnes, Providencia spp, Pseudomonas aeruginosa, Orientia, Ruminococcus bromii, Rothia dentocariosa, Ruminococcus spp, Sarcinalutea spp., Serratia marcescens, Shigella boydii, Shigella fiexneri, Shigella sonnei, Sarcina spp, Staphylococcus aureus, Staphylococcus epidermidis, Streptococcus anginosus, Streptococcus faecalis, Streptococcus mutans, Streptococcus oxalis, Streptococcus pneumoniae, Streptococcus sobrinus, Streptococcus viridans, Streptococcus pyogenes, Salmonella, Salmonella typhi, Salmonella paratyphi, Rickettsi, Torulopsis glabrata, Treponema denticola, Treponema refringens, Veillonella spp, Vibrio spp, Vibrio sputorum, Wolinella succinogenes, Yersinia enterocolitica, or any combination thereof.

In some aspects, one or more of the plurality of bacteria species are pathogenic bacteria. In some aspects, the pathogenic bacteria can be Clostridium difficile, Salmonella spp., enteropathogenic E. coli, multi-drug resistant bacteria such as Klebsiella, and E. coli, Carbapenem-resistant Enterobacteriaceae (CRE), extended spectrum beta-lactam resistant Enterococci (ESBL), fluoroquinolone-resistant Enterobacteriaceae, and vancomycin-resistant Enterococci (VRE), multi-drug resistant bacteria, extended spectrum beta-lactam resistant Enterococci (ESBL), Carbapenem-resistant Enterobacteriaceae (CRE), fluoroquinolone-resistant Enterobacteriaceae, and vancomycin-resistant Enterococci (VRE), Aeromonas hydrophila, Campylobacter fetus, Plesiomonas shigelloides, Bacillus cereus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enterotoxigenic Escherichia coli (such as, but not limited to, LT and/or ST), Escherichia coli O157:H7, Helicobacter pylori, Klebsiella pneumonia, Lysteria monocytogenes, Plesiomonas shigelloides, Salmonella spp., Salmonella typhi, Salmonella paratyphi, Shigella spp., Staphylococcus spp., Staphylococcus aureus, vancomycin-resistant enterococcus spp., Vibrio spp., Vibrio cholerae, Vibrio parahaemolyticus, Vibrio vulnificus, and Yersinia enterocolitica, antibiotic-resistant Proteobacteria, Vancomycin Resistant Enterococcus (VRE), Carbapenem Resistant Enterobacteriaceae (CRE), fluoroquinolone-resistant Enterobacteriaceae, Extended Spectrum Beta-Lactamase producing Enterobacteriaceae (ESBL-E), or any combination thereof.

In some aspects, one or more of the plurality of bacteria species can be antibiotic resistant bacteria including, but not limited, to Acinetobacter baumannii, Enterobacter cloacae, Escherichia coli, Klebsiella aerogenes, Klebsiella pneumoniae, Pseudomonas aeruginosa, Salmonella enterica, Staphylococcus aureus, Streptococcus pneumoniae, Klebsiella oxytoca, Serratia marcescens, Enterobacter aerogenes, Proteus mirabilis, Acinetobacter baumannii, Stenotrophomonas maltophilia, Staphylococcus epidermidis, Staphylococcus haemolyticus, Staphylococcus saprophyticus, Streptococcus pyogenes, Streptococcus agalactiae, Streptococcus mitis, Enterococcus faecium, Enterococcus faecalis, Candida albicans, Candida tropicalis, Candida parapsilosis, Candida krusei, Candida glabrata, Mycobacterium tuberculosis, Neisseria meningitidis, Listeria monocytogenes, Citrobacter freundii, Salmonella enteritidis, Serratia marcescens, Proteus mirabilis, Hafnia alvei, Enterobacter spp, Serratia marcescens, Pseudomonas putida, Enterobacter cloacae, Proteus vulgaris, Providencia rettgeri, Shigella flexneri, Shewanella algae, Acinobacter junii, Ralstonia pickettii, Pandoraea pnomenusa, Pasteurella multocida, Bordetella bronchiseptica, Listeria monocytogenes, Bacillus cereus, or any combination thereof.

In some aspects, one or more of the plurality of bacteria species can be multidrug resistant. Multi-drug resistant bacteria may include, but are not limited to, Acinetobacter Baumannii such as ATCC isolate #2894233-696-101-1, ATCC isolate #2894257-696-101-1 ATCC isolate #2894255-696-101-1, ATCC isolate #2894253-696-101-1, or ATCC #2894254-696-101-1; Citrobacter freundii such as ATCC isolate #33128, ATCC isolate #2894218-696-101-1, ATCC isolate #2894219-696-101-1, ATCC isolate #2894224-696-101-1, ATCC isolate #2894218-632-101-1, or ATCC isolate #2894218-659-101-1; Enterobacter cloacae such as ATCC isolate #22894251-659-101-1, ATCC isolate #22894264-659-101-1, ATCC isolate #22894246-659-101-1, ATCC isolate #22894243-659-101-1, or ATCC isolate #22894245-659-101-1; Enterococcus facilis such as ATCC isolate #22894228-659-101-1 ATCC isolate #22894222-659-101-1, ATCC isolate #22894221-659-101-1, ATCC isolate #22894225-659-101-1, or ATCC isolate #22894245-659-101-1; Enterococcus faecium such as ATCC isolate #51858, ATCC isolate #35667, ATCC isolate #2954833_2694008 ATCC isolate #2954833_2692765, or ATCC isolate #2954836_2694361; Escherichia coli such as ATCC isolate CGUC 11332, CGUC 11350, CGUC 11371, CGUC 11378, or CGUC 11393; Klebsiella pneumonia such as ATTC isolate #27736, ATTC isolate #29011, ATTC isolate #20013, ATTC isolate #33495, or ATTC isolate #35657; Serratia marcescens such as ATCC isolate #43862, ATCC isolate #2338870, ATCC isolate #2426026, ATCC isolate #SIID 2895511, or ATCC isolate #SIID 2895538; or Staphylococcus aureus such as ATCC isolate #JHH 02, ATCC isolate #JHH 02, ATCC isolate #JHH 03, ATCC isolate #JHH 04, ATCC isolate #JHH 05, or ATCC isolate #JHH 06.

In some aspects, the machine learning algorithm is trained using bacteria genetic information associated with an antibiotic resistant bacteria. In some aspects, the machine learning algorithm is trained using genetic information associated with Acinetobacter baumannii, Enterobacter cloacae, Escherichia coli, Klebsiella aerogenes, Klebsiella pneumoniae, Pseudomonas aeruginosa, Salmonella enterica, Staphylococcus aureus, Streptococcus pneumoniae, or any combination thereof. In some aspects, the machine learning algorithm is trained using genetic information associated with Acinetobacter baumannii. In some aspects, the machine learning algorithm is trained using genetic information associated with Enterobacter cloacae. In some aspects, the machine learning algorithm is trained using genetic information associated with Escherichia coli. In some aspects, the machine learning algorithm is trained using genetic information associated with Klebsiella aerogenes. In some aspects, the machine learning algorithm is trained using genetic information associated with Klebsiella pneumoniae. In some aspects, the machine learning algorithm is trained using genetic information associated with Pseudomonas aeruginosa. In some aspects, the machine learning algorithm is trained using genetic information associated with Salmonella enterica. In some aspects, the machine learning algorithm is trained using genetic information associated with Staphylococcus aureus. In some aspects, the machine learning algorithm is trained using genetic information associated with Acinetobacter baumannii, Enterobacter cloacae, Escherichia coli, Klebsiella aerogenes, Klebsiella pneumoniae, Pseudomonas aeruginosa, Salmonella enterica, Staphylococcus aureus, and Streptococcus pneumoniae.

In some examples, the antibiotic resistance prediction system(s) 200 and 500-1600 can perform various operations to derive genetic features.

For examples, as noted above, the antibiotic resistance prediction system(s) 200 and 500-1600 can derive explainable KEGG ortholog gene-based sequence variants. The sequence reads can be assembled to obtain consensus reference genomes, the gene sequences can be detected and matched with clustered UniRef20 protein sequences, and the different reference gene clusters can be associated to the KEGG ortholog (KO) genes. For example, the variant K18768.0 can indicate blaKPC, the K. pneumoniae carbapenemase. Here the K18768 is the KEGG KO gene name and 0 represents the UniRef cluster. Amino-acid features can lead to good prediction performance in single combinations of bacterial species and antibiotics.

In some examples, the PARP model 504 can have various model architectures and parameters.

For example, the PARP model 504 can be built from at least four types of blocks, as shown in FIG. 3. These blocks can include a Variant block (Var block) to calculate the embedding of bacterial variants; a Dense block to represent variant-level features using deep neural networks; a FiLM block, consisting of a FiLM Generator to transform antibiotic features and blend bacterial variant features through conditional affine transformation, thus effectively blending the features from both domains; and/or a Classifier block to calculate the probability of being resistant or susceptible. The hyperparameter(s) of the model of these blocks can be tuned using grid search. Specifically, the geometric size of the dense layer can be tuned (e.g., 64, 128, 256, 512, or 1024), the number of Dense Blocks can be tuned (e.g., 1, 2, or 3), and/or the number of FiLM Generators can be tuned (e.g., 1, 2, 3, 4, 5, 6, or 7).

In some scenarios, the antibiotic resistance prediction system(s) 200 and 500-1600 can determine an optimized set of parameters using the nested cross-validation procedure 1202 of FIGS. 12A and 12B to report unbiased prediction accuracies. For instance, as discussed above regarding FIGS. 12A and 12B, the whole data set (e.g., N=29,187) can first be split into three outer folds, where two outer folds serve as training and the rest as testing. Next, the training set can be split into three inner folds, where two inner folds serve as sub-training and the rest as validation. Using each hyperparameter combination, a neural network can be trained in 10 epochs in the sub-training set. The hyperparameters can be retrained with the largest validation accuracy and used to retrain a neural network on training samples for each outer fold. The mean accuracies over the three outer folds as the overall prediction accuracy can be reported, defined as:

$$Accuracy = {\sum }_{i = 1}^{93} \frac{{s}_{i 1} + {s}_{i 2} + {s}_{i 3}}{3} . {p}_{i}$$

where sᵢⱼ is the outer test accuracy on the test set of j^{th} outer folder for the i^{th} bacteria-antibiotic combination and pᵢ is the proportion of i^{th} combination in the training dataset. Similarly, the area below AUROC as the mean AUROC over outer folds weighted by the proportional weights of the bacteria-antibiotic combination can be reported.

In some examples, the antibiotic resistance prediction system(s) 200 and 500-1600 can generate one or more model explanations.

For example, to understand the structure inside the PARP model 504, the antibiotic resistance prediction system(s) 200 and 500-1600 can analyze the estimated parameters from the “hidden” blocks (e.g., neural network layers) inside the model. The antibiotic resistance prediction system(s) 200 and 500-1600 can employ at least two unsupervised learning algorithms, such as a principal component analysis (PCA) and/or a hierarchical cluster analysis (HCA). The PCA can project original data to the principal component space, which can be a low-dimensional feature set preserving the original data variation at best effort. Similar observations can be clustered together in the lower-dimensional space. Additionally or alternatively, the HCA can seek homogeneous subgroups among the original observations by iteratively fusing two clusters sharing the most similarity. To interpret the 29 antibiotics, the antibiotic resistance prediction system(s) 200 and 500-1600 can average the feature maps of dense layers from all the FiLM Generators to obtain a weight matrix W_{FILM}∈R29×1,024. For the PCA visualization, the antibiotic resistance prediction system(s) 200 and 500-1600 can project the 29 observations into a 2-dimensional space using the first two principal components calculated by the PCA decomposition function in the sklearn package. For the HCA, the antibiotic resistance prediction system(s) 200 and 500-1600 can use the agglomerative clustering method from a cluster function in the sklearn package to obtain the hierarchical clustering results for all antibiotics. Similarly, the antibiotic resistance prediction system(s) 200 and 500-1600 can use the weights W_{Var}∈R 3,393×1,024 from the dense layer located in the Var Block and directly downstream of the input genetic variants. The antibiotic resistance prediction system(s) 200 and 500-1600 can perform PCA and HCA on W_{Var}, representing the information the PARP model 504 learned from the 3,393 unique bacteria isolates.

In addition, the contribution of each genetic mutation to resistance conditioned on a wild-type baseline through model agnostic explanation can be estimated. An indicator vector of length 14,615 as the bacterial genetic feature input can be created manually, and one-hot encoded antibiotics as the antibiotic feature input can be used. The PARP model 504 can output the probability of resistance as the baseline, p⁰_{resistance}. Then, the antibiotic resistance prediction system(s) 200 and 500-1600 can mutate the i^{th} element to 1 to mimic a bacterium isolate carrying the corresponding variant. With this mutated genetic feature vector, the PARP model 504 can compute the new probability of resistance, pⁱ_{resistance}. The PARP model 504 can use the difference, Effectᵢ=pⁱ_{resistance}−p⁰_{resistance}, to represent the effect of the genetic variant i for a given antibiotic.

In some examples, the antibiotic resistance prediction system(s) 200 and 500-1600 can generate one or more predictions for unseen bacteria and antibiotics combinations.

For example, the PARP model 504 can be a unified AR prediction model for multiple bacteria-antibiotic combinations and, as such, it can have the potential to predict resistance probabilities for novel bacteria-antibiotic combinations by leveraging the information learned from existing combinations. To quantitively assess its performance, the antibiotic resistance prediction system(s) 200 and 500-1600 can conduct a leave-one-combination-out (LOCO) experiment by excluding isolates from one bacterium-antibiotic combination, rebuilding the PARP model 504 on the remaining training data, and predicting AR on the holdout isolates. Also, the prediction accuracy for each LOCO experiment can be reported based on the PARP model architecture.

Next, in some scenarios, the LOCO models can be evaluated on an external dataset, for instance, containing 1,075 samples from 18 various bacteria-antibiotics pairs. For each specific bacterium-antibiotic pair in the external dataset, the antibiotic resistance prediction system(s) 200 and 500-1600 can select the model trained in the above LOCO experiment, where the samples from this pair were excluded. Then, the model performance on samples belonging to this bacterium-antibiotic pair can be evaluated. A high accuracy of 99.26% can be reached for Pseudomonas aeruginosa with respect to amikacin, which can indicate that the PARP model 504 could predict some bacteria-antibiotics pairs which do not exist in the training dataset.

In some examples, the antibiotic resistance prediction system(s) 200 and 500-1600 performs various external validation procedures.

For example, to validate the prediction performance of the PARP model 504, the antibiotic resistance prediction system(s) 200 and 500-1600 can use the external dataset collected at MD Anderson. As noted above, the PARP model 504 can be trained using the dataset (N=29,187) having an optimal hyperparameter set (e.g., one Dense Block, two FiLM Generators, and/or a dense layer size of 1,024) and can use 30 epochs and batch size 32. Performance metrics can include the overall prediction accuracy, receiver operating characteristic (ROC) curves, and/or AUROC values for individual bacteria-antibiotic combination. The overall prediction accuracy can be the weighted average prediction accuracy of an individual bacteria-antibiotic pair:

$$overall prediction accuracy = {\sum }_{i = 1}^{21} {a}_{i} . {p}_{i} ,$$

where aᵢ is the prediction accuracy on the i^{th} bacteria-antibiotic pair and pᵢ is the corresponding proportion in the external dataset. Along with the AUROC value, this can be a powerful tool to speculate binary classification models. It can depict relative trade-offs between sensitivity and specificity for thresholds ranging from 0 to 1.

In some examples, the PARP model 504 can be trained end-to-end from scratch with batch size 32, an RMSprop optimizer with a learning rate of 0.001, a ReLU activation, 10 epochs, and/or a dropout rate of 0.5. The test size proportion in the outer fold can be 33% and the validation size proportion in the inner fold can be 20% during the nested cross-validation procedure 1202.

In some scenarios, the PARP model 504 for pan-antibiotic resistance prediction is based on the techniques of deep learning models that can sometimes be criticized as black-boxed. To enhance interpretability, the PARP model 504 can be designed explicitly, as each network block has its purpose. Employing the FiLM structure can efficiently blend the bacterial genetic and antibiotic features. The network parameters can also be optimized to visualize the PARP model 504. Accordingly, an explanation of the model can be provided to improve users' understanding. of the outputs. Furthermore, the PARP model 504 can predict untrained bacteria-antibiotic combinations. The PARP model 504 can be used for under-represented combinations or when sample size is a concern.

In some instances, while proteins are the main functional units in prokaryotic organisms which contribute to common resistance mechanisms, there could be other mechanisms (e.g., metabolism related genes) associated with resistance that genomics alone will not capture, which can be used by the antibiotic resistance prediction system(s) 200 and 500-1600 to enlarge the gene features based on the PARP model 504. As such, the PARP model 504 can be a useful tool for predicting antibiotic resistance across pathogen space using a variety of different mechanisms.

As discussed, this disclosure provides method for predicting antibiotic resistance using a plurality of antibiotics. The plurality of antibiotics (e.g., two or more) may include antibiotics known in the art. Similarly, the antibiotic resistance that is predicted by the disclosed methods, may be an antibiotic known in the art.

In some aspects, the antibiotic(s) may be a macrolide antibiotic, sulfa antibiotic, carbostyril antibiotic, nitrofuran antibiotic, cephalosporin analog, or any combination thereof.

In some aspects, the antibiotic(s) of the present disclosure may be from a class of antibiotics, non-limiting examples of antibiotic classes include aminoglycosides, carbapenems and monobactams, cephalosporins, chloramphenicol, lincosamides, macrolides, pleuromutilins, glycopeptides, polypeptides, penicillins, polymixins, quinolones, sulfonamides and tetracyclines, among others. In some aspects, the antibiotic(s) can comprise penicillin (e.g., ampicillin, piperacillin, benzylpenicillin, methicillin, and cloxacillin), cephalosporin (for e.g., cefotaxime and ceftazidime, cephaloridine), carbapenem (e.g., iminipenen, meropenem, etrapenem, doripenem), monobactam (e.g., aztreonam), or any combination thereof.

In some aspects, the antibiotic(s) may be gentamicin, kanamycins, streptomysin, neomycin, tetracycline, terramycin, aureomycin, doxycycline, erythromycin, roxithromycin, sulphadiazine, sulfadimidine, sulfadimethoxine, sulfamethoxazole, sulfadoxine, norfloxacin, ciprofloxacin, ofloxacin, gatifloxacin, sparfloxacin, moxifloxacin, furazolidone, furaltadone, furantoin, nitrofurazone, chloromycetin, thiamphenicol, clindamycin, lincomycin, ampicillin, gentamicin, kanamycin, streptomycin, erythromycin, clindamycin, tetracycline, chloramphenicol, balofloxacin, ceftiofur, cinoxacin, ciprofloxacin, clinafloxacin, enoxacin, fleroxacin, gemifloxacin, levofloxacin, lomefloxacin, nadifloxacin, nalidixic acid, oxolinic acid, pazufloxacin, pefloxacin, pipemidic acid, piromidic acid, prulifloxacin, rosoxacin, rufloxacin, sitafloxacin, sparfloxacin, tosufloxacin, chlortetracycline, demeclocycline, doxycycline, lymecycline, meclocycline, methacycline, minocycline, omadacycline, oxytetracycline, rolitetracycline, sarecycline, amikacin, cefepime, imipenem, amoxicillin, amoxicillin/clavulanate, ampicillin/sulbactam, azithromycin, cefalothin, cefazolin, cefepime, cefotaxime, cefoxitin, ceftriaxone, cefuroxime, daptomycin, ertapenem, fosfomycin, fusidic acid, linezolid, meropenem, methicillin, mupirocin, nitrofurantoin, oxacillin, penicillin, piperacillin/tazobactam, quinupristin/dalfopristin, rifampicin, teicoplanin, tigecycline, tobramycin, trimethoprim/sulfamethoxazole, vancomycin, or any combination thereof.

In some aspects, the antibiotic(s) comprise amoxicillin, meropenem, amoxicillin/clavulanic, cefoxitin, chloramphenicol, kanamycin, trimethoprim/sulfamethoxazole, ceftiofur, ciprofloxacin, ceftazidime, ampicillin, cefotaxime, ampicillin/sulbactam, aztreonam, ceftriaxone, tetracycline, ertapenem, erythromycin, tobramycin, amikacin, clindamycin, cefazolin, levofloxacin, doripenem, impipenem, gentamicin, cefepime, cefuroxime, piperacillin/tazobactam, or any combination thereof. In some aspects, the antibiotic(s) comprise amoxicillin, meropenem, amoxicillin/clavulanic, cefoxitin, chloramphenicol, kanamycin, trimethoprim/sulfamethoxazole, ceftiofur, ciprofloxacin, ceftazidime, ampicillin, cefotaxime, ampicillin/sulbactam, aztreonam, ceftriaxone, tetracycline, ertapenem, erythromycin, tobramycin, amikacin, clindamycin, cefazolin, levofloxacin, doripenem, impipenem, gentamicin, cefepime, cefuroxime, piperacillin/tazobactam, linezolid, tidezolid, ceftazidime-avibactam, ceftolozane-tazobactam, cefiderocol, imipenem-relebactam, durlobactam-sulbactam, fidaxomicin, eravacycline, dalbavancin, and ceftaroline.

In some aspects, the antibiotic(s) comprise amikacin, ampicillin, cefepime, linezolid, tidezolid, ceftazidime-avibactam, ceftolozane-tazobactam, cefiderocol, imipenem-relebactam, durlobactam-sulbactam, fidaxomicin, eravacycline, dalbavancin, ceftaroline, or any combination thereof. In some aspects, the antibiotic is amikacin. In some aspects, the antibiotic is ampicillin. In some aspects, the antibiotic is cefepime. In some aspects, linezolid. In some aspects, the antibiotic is tidezolid. In some aspects, the antibiotic is ceftazidime-avibactam. In some aspects, the antibiotic is ceftolozane-tazobactam. In some aspects, the antibiotic is cefiderocol. In some aspects, the antibiotic is imipenem-relebactam. In some aspects, the antibiotic is durlobactam-sulbactam. In some aspects, the antibiotic is fidaxomicin. In some aspects, the antibiotic is eravacycline. In some aspects, the antibiotic is dalbavancin. In some aspects, the antibiotic is ceftaroline.

While specific implementations are discussed, it should be understood that this is done for illustration purposes only. A person skilled in the relevant art will recognize that other components and configurations may be used without parting from the spirit and scope of the presently disclosed technology. Thus, the following description and drawings are illustrative and are not to be construed as limiting. Numerous specific details are described to provide a thorough understanding of the presently disclosed technology. However, in certain instances, well-known or conventional details are not described in order to avoid obscuring the description. References to one or an implementation in the presently disclosed technology can be references to the same implementation or any implementation; and such references mean at least one of the implementations.

Reference to “one implementation” or “an implementation” means that a particular feature, structure, or characteristic described in connection with the implementation is included in at least one implementation of the presently disclosed technology. The appearances of the phrase “in one implementation” in various places in the specification are not necessarily all referring to the same implementation, nor are separate or alternative implementations mutually exclusive of other implementations. Moreover, various features are described which may be exhibited by some implementations and not by others.

The terms used in this specification generally have their ordinary meanings in the art, within the context of the presently disclosed technology, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the presently disclosed technology or of any example term. Likewise, the presently disclosed technology is not limited to various implementations given in this specification.

Without intent to limit the scope of the presently disclosed technology, examples of instruments, apparatus, methods and their related results according to the implementations of the presently disclosed technology are given below. Note that titles or subtitles may be used in the examples for convenience of a reader, which in no way should limit the scope of the presently disclosed technology. Unless otherwise defined, technical and scientific terms used herein have the meaning as commonly understood by one of ordinary skill in the art to which the presently disclosed technology pertains. In the case of conflict, the present document, including definitions will control.

Additional features and advantages of the presently disclosed technology will be set forth in the description which follows, and in part will be obvious from the description, or can be learned by practice of the herein disclosed principles. The features and advantages of the presently disclosed technology can be realized and obtained by means of the instruments and combinations particularly pointed out in the appended claims. These and other features of the presently disclosed technology will become more fully apparent from the following description and appended claims or can be learned by the practice of the principles set forth herein.

What is claimed is:

1. A method for antibiotic resistance prediction comprising:
receiving, at a machine learning system, genetic information associated with a bacteria, a species of the bacteria being one of a plurality of bacterial species;
receiving, at the machine learning system, an indication of an antibiotic of a plurality of antibiotics, wherein the machine learning system is trained using genetic information associated with the plurality of bacterial species and for the plurality of antibiotics; and
outputting, at the machine learning system, an indication of an antibiotic resistance associated with the bacteria and the antibiotic received at the machine learning system.

2. The method of claim 1, wherein the machine learning system is trained using protein sequences associated with the plurality of bacterial species.

3. The method of claim 1, wherein the machine learning system comprises a feature-wise linear modulation (FiLM) machine learning system.

4. The method of claim 1, wherein the machine learning system jointly models antibiotics and bacterial variants.

5. The method of claim 1, wherein the machine learning system includes rectified linear activation function (ReLU) layer, a batch normalization layer, and a dropout layer.

6. A method for antibiotic resistance prediction comprising:
training a pan-antibiotic resistance prediction (PARP) model by providing a machine learning system with one or more training data sets including:
genetic information associated with a plurality of bacterial species, and
antibiotic feature information associated with a plurality of antibiotics;
receiving, at the machine learning system, a genomic sequence associated with a particular bacterial isolate; and
outputting, at the machine learning system, a predictive indication of an antibiotic resistance, associated with one or more antibiotics, for the particular bacterial isolate.

7. The method of claim 6, further comprising:
performing a data preparation procedure on the one or more training data sets by one-hot encoding the antibiotic feature information.

8. The method of claim 6, wherein the one or more training data sets include at least one of an isolates-variants matrix, an antibiotics indicator matrix, or an isolates resistance symptom vector.

9. The method of claim 6, further comprising:
performing a nested cross-validation procedure on the PARP model using a validation data set including a plurality of bacteria-antibiotic combinations.

10. The method of claim 6, further comprising:
determining, with the PARP model, one or more classes associated with the plurality of antibiotics, or the plurality of bacterial species, using weights of one or more dense layers of a Feature wise Linear Modulator (FiLM) generator to form clusters,
wherein the machine learning system uses the one or more classes to output the predictive indication of the antibiotic resistance.

11. The method of claim 6, wherein the PARP model is deployed onto a container orchestration service such that the PARP model provides a cloud-based antibiotics resistance prediction service.

12. The method of claim 11, wherein receiving the genomic sequence includes receiving an upload, from a remote device, at the cloud-based antibiotics resistance prediction service.

13. The method of claim 6, wherein the genomic sequence corresponds to a bacterial species absent from the one or more training data sets.

14. The method of claim 6, wherein the predictive indication of the antibiotic resistance includes a bar graph for presentation at a graphical user interface (GUI) of a computing device that provided the genomic sequence.

15. The method of claim 14, wherein an x-axis of the bar graph represents different antibiotics and a y-axis of the bar graph represents a prediction value of resistance or susceptibility to the different antibiotics.

16. The method of claim 6, wherein the PARP model generates shared and unique variant data indicating one or more variants shared between different bacteria species and one or more variants unique to the different bacteria species.

17. The method of claim 6, wherein training the PARP model includes generating paired-antibiotic susceptibility data based on tests of isolates on antibiotic pairs indicating shared pathways of the antibiotic pairs.

18. The method of claim 6, further comprising:
performing a prediction accuracy assessment for the predictive indication, the prediction accuracy assessment outputs one or more prediction accuracy values corresponding to the one or more antibiotics.

19. The method of claim 6, further comprising:
tuning a plurality of hyperparameters of the PARP model, the plurality of hyperparameters includes:
a number of dense blocks,
a number of FiLM layers or feature stacking blocks, and
a geometric size of a dense layer.

20. A system for antibiotic resistance prediction comprising:
a pan-antibiotic resistance prediction (PARP) model deployed to a cloud-based service, the PARP model having a machine learning system trained with one or more training data sets including:
genetic information associated with a plurality of bacterial species, and
antibiotic feature information associated with a plurality of antibiotics;
a web-based portal for receiving a genomic sequence associated with a particular bacterial isolate and providing the genomic sequence to the PARP model; and
a predictive indication of an antibiotic resistance, for the particular bacterial isolate, outputted by the PARP model and configured for presentation at a graphical user interface (GUI) of a computing device.
